(19) United States
(12) Patent Application Publication
(10) Pub. No.: US 2026/0026733 A1
(43) Pub. Date: Jan. 29, 2026

(54) SYSTEM FOR PREDICTING A PROGNOSIS OF THE NEUROPSYCHOLOGICAL AND/OR NEUROPSYCHIATRIC STATUS IN A SUBJECT

(71) Applicant: ECOLE POLYTECHNIQUE FEDERALE DE LAUSANNE (EPFL), Lausanne (CH)

(72) Inventors: Fosco BERNASCONI, Nyon (CH); Olaf BLANKE, Nyon (CH)

(21) Appl. No.: 19/127,963

(22) PCT Filed: Nov. 9, 2023
(86) PCT No.: PCT/IB2023/061329

§ 371 Date: May 7, 2025

(30) Foreign Application Priority Data
Nov. 9, 2022 (WO) PCT/IB2022/060771

(51) Int. Cl. A61B 5/374; A61B 5/00

(52) U.S. Cl. CPC A61B 5/374; A61B 5/4088; A61B 5/7275

(57) ABSTRACT

The present invention relates to systems and methods for predicting a prognosis of the neuropsychological and/or neuropsychiatric status in a subject based on reports of Minor Hallucination (MH) events in combination with electrophysiological data of the subject.

### TECHNICAL FIELD

The present invention generally belongs to the fields of neuropsychology and neuropsychiatry. In particular, the invention relates to systems and methods for predicting a prognosis of the neuropsychological and/or neuropsychiatric status in a subject based on reports of Minor Hallucination (MH) events in combination with electrophysiological data of the subject.

### BACKGROUND ART

Neurological disorders strike an estimated 50 million Americans each year, exacting an incalculable personal toll and an annual economic cost of hundreds of billions of dollars in medical expenses and lost productivity. The burden of neurological disease is a burden borne by every segment of society, and people everywhere. Indeed, individuals suffering from disorders such as neuropsychological and/or neuropsychiatric ones often require care 24 hours a day, seven days a week. In such cases family members from several households may need to partake in the care, and these caregivers usually have jobs, as well as their own families. Thus, the consequences of neurological disease have far reaching consequences.

Among neurological disorders, neuropsychiatric and neurodegenerative diseases are a major clinical problem that may manifest in a number of forms such as frontotemporal lobar degeneration, Alzheimer's disease, Motor Neuron Disease, Lewy body diseases, Parkinson Disease, REM sleep behaviour disorder (RBD), major depression or schizophrenia, to cite a few.

Many of these neuropsychiatric or neurodegenerative diseases are often accompanied by dementia. Dementia is the progressive decline in cognitive function due to damage or disease in the brain beyond what might be expected from e.g. normal aging. Particularly affected areas may be memory, attention, language, and problem solving. Especially in the later stages of the condition, affected persons may be disoriented in time (not knowing what day of the week, day of the month, month, or even what year it is), in place (not knowing where they are), and in person (not knowing who they are). The prevalence of dementia is rising as the global life expectancy is rising. Particularly in Western countries, there is increasing concern about the economic impact that dementia will have in future older populations. Dementia is a nonspecific term encompassing many disease processes, including cognitive decline or impairment. At present there is no cure for any type of dementia.

Parkinson's disease (PD) affects approximately 3% of the population over 65 years of age and the number of patients is expected to double by 2040, reaching an estimated total of 15-18 million people worldwide. Although PD is traditionally defined as a movement disorder with the typical symptoms of resting tremor, rigidity, and bradykinesia, there is now ample evidence that the PD is also affecting several non-motor circuits leading to a wide variety of non-motor symptoms. Among the non-motor symptoms hallucinations are highly prevalent, with one individual out of two experiencing hallucinations regularly, and at an advanced stage of the disease, the occurrence of hallucinations may increase up to 70%, often becoming the dominant non-motor symptom, together with dementia, beyond the well-known motor symptoms.

Hallucinations in PD are of major negative impact on patients, families, and society, and may indicate a more severe form of the disease, characterized by chronic psychosis, delusions, more rapid cognitive decline, and dementia (PD dementia, PDD). Hallucinations also increase the likelihood of earlier home placement and are associated with a higher mortality.

Hallucinations are often categorized into formed (well-structured, formed, or complex) visual hallucinations (VH) and so-called minor hallucinations (MH), which include presence and passage hallucinations, and pareidolias. VH generally occur at the middle to late stage of the disease, and several studies have identified VH as a risk factor for PPD. However, because VH occur at a more advanced stage of the disease, with cognitive decline already present, they are not suitable as an early marker of cognitive decline in PD. This seems to differ for MH, which are usually experienced at earlier stages of the disease, and can even precede parkinsonian motor symptoms, testifying to the importance to include them in detailed clinical evaluations. Recent data show that MH are not only the earliest hallucination occurring in PD, but they also share brain alterations with VH and are linked to early cognitive deficits, underlining their potential role as a marker for PDD. Yet, despite their high prevalence and likely association with major negative clinical outcome, MH remain highly understudied and their neural mechanisms and association with cognitive decline and PDD poorly understood.

Clinical evidence suggests the importance to classify cognitive dysfunction in PD into two distinctive subtypes: impairments in frontal-subcortical (executive) functions and impairments in posterior-cortical (visuo-spatial) deficits. To assess deficits in frontal-subcortical and/or posterior cognitive functions specific screening instruments have been developed, such as the Parkinson's Disease-Cognitive Rating Scale [PD-CRS]. The PD-CRS separates frontal-subcortical executive tasks (e.g. working memory, verbal fluency) and posterior cortical visuo-spatial tasks (e.g. clock copying). While posterior-cortical deficits have been argued to be important to signal the transition from mild cognitive impairments to PDD, frontal-subcortical deficits have been deemed characteristic of earlier phases of PD cognitive impairment.

Next to neuropsychology and hallucinations, electroencephalography (EEG) recordings have been explored for cognitive decline assessment in different neurological disorders. EEG recording systems are available in most hospitals and clinics (even at patient homes), and allow for measuring brain activity, oscillations, and function(s). In PD, analysis of neural oscillations has revealed motor-related changes, such as enhanced beta oscillations in subcortical (e.g. subthalamic nucleus) and between subcortical structures and the motor cortex. Concerning cognitive decline, the EEG of patients with PD has revealed an enhancement of theta oscillatory power, as well as a reduction in the higher frequency bands, depending on cognitive decline (cross-sectional studies). In addition, it has been suggested that especially enhanced theta oscillations might indicate an increased risk of PDD. Despite the putative role of theta oscillations in cognitive decline, it not known which feature might underline those changes. For instance, it is not known whether and how these cognition-related oscillatory changes relate to hallucinations (in particular MH), and how the blending of those features might allow an earlier and more accurate identification of patients at higher risk of cognitive decline. To gather and validate those information, a continuous and systematic longitudinal evaluation of the patients is necessary.

In order to determine the evolution of the neuropsychological and/or neuropsychiatric status in a subject, and in defining which subjects have a higher risk to undergo a degradation in cognitive performances such as cognitive decline or dementia, or even to undergo certain psychotic conditions (due for instance to occurrence of major hallucinations), it is highly desirable to establish and implement robust and reliable systems and methods assessing several parameters, either of neurophysiological and neuropsychiatric nature. The present invention addresses and overcomes at least some of the current limitations in this field.

### SUMMARY OF INVENTION

According to the above aims, a main purpose of the present invention is that of determining the likelihood of a subject to undergo a cognitive decline or degradation of cognitive functions based on the assessment of neurophysiological and neuropsychiatric parameters.

Still another purpose of the present invention is that of providing an early identification of a more severe form of neurological or neurodegenerative disorder, such as PD, associated with cognitive decline and psychosis.

These objectives have been accomplished with the present invention, as described herein and in the appended claims.

In view of the above-summarized drawbacks and/or problems affecting the prior art solution, a first object of the present invention is that of providing a system for predicting a prognosis of the neuropsychological and/or neuropsychiatric status in a subject, according to claim 1.

In particular, an object of the invention relates to a system for predicting a prognosis of a neuropsychological and/or a neuropsychiatric status in a subject, the system comprising a first device (or a first measuring device or a first input device) for measuring or obtaining data regarding the neuropsychiatric and/or neuropsychological status of a subject at a time T₀, said data comprising information related to Minor Hallucination (MH) events. The system also comprises a second measuring device for measuring electrophysiological data of the subjects at a time T₀, said electrophysiological data comprising frontal theta oscillatory frequency band power and/or activity; and a data analyzing device for gathering and analyzing the data regarding the neuropsychiatric and/or neuropsychological status and the electrophysiological data and comparing the data to a reference, wherein a report or a presence of Minor Hallucination (MH) events, together with an enhanced frontal theta oscillatory frequency band power and/or activity is indicative of a negative prognosis of the neuropsychological and/or neuropsychiatric status in the subject.

Frontal theta oscillatory frequency may be comprised for example between 4 Hz to 12 Hz or between 4 Hz to 8 Hz.

In an embodiment, the system may comprise a memory device for storing the data regarding the neuropsychiatric and/or neuropsychological status of the subject and the electrophysiological data.

In one embodiment, the first device comprises or consists of a robotic master-slave system configured for inducing a Minor Hallucination (MH).

In an embodiment, the robotic master-slave system is configured for inducing Presence Hallucination (PH) such as a Feeling of a Presence (FoP) hallucination.

The robotic system preferably includes or comprises:
(a) a master device adapted to be moved, moved on or manipulated by a subject; and
(b) a slave device operably connected with the master device and adapted so that the subject is directly or indirectly touched by the slave device according with the master device's movement.

In one embodiment, the robotic system further comprise (c) a computer device operably connected to both the master and the slave device, the computer device configured to:
modulate the time and/or space and/or force activation of the slave device in response to the activation of the master device.

In one embodiment, the computer device is further configured to:
record data regarding the difference in time and/or space and/or force activation;
comparing the recorded data with reference data; and
graphically or numerically showing the result of the comparison on a display means, for example a display screen of a computer.

In one embodiment, the computer device modulates the activation of the slave device over time.

In one embodiment, the computer device modulates the activation of the slave device so to create a temporal mismatch between the master device activation and the slave device response over time.

A second object of the present invention is that of providing a method for predicting a prognosis of the neuropsychological and/or neuropsychiatric status in a subject, according to claim 14.

In particular, an object of the invention relates to a method for predicting a prognosis of the neuropsychological and/or neuropsychiatric status in a subject, the method comprising the steps of:
i) obtaining data regarding the neuropsychiatric status of the subject at a time T₀, said data comprising information related to Minor Hallucination (MH) events; and
ii) obtaining electrophysiological data of the subject at a time T₀ comprising frontal theta oscillatory frequency band power and/or activity,
wherein the report or the presence of Minor Hallucination (MH) events, together with an enhanced frontal theta oscillatory frequency band power and/or activity compared to a reference, is indicative of a negative prognosis of the neuropsychological and/or neuropsychiatric status in the subject.

Advantageously, the subject is a patient suffering from a neurological disease and/or neuropsychiatric disease selected from a list comprising Alzheimer's disease, Parkinson's disease (PD), Dementia with Lewy bodies (DLB), REM sleep behaviour disorder (RBD), major depression, schizophrenia.

In one embodiment, Minor Hallucination (MH) events include sense of presence, passage hallucinations, and/or visual illusions, and/or Presence Hallucination (PH), and/or Feeling of a Presence (FoP) Hallucination.

In one embodiment, information related to Minor Hallucination (MH) events are obtained through at least one of MDS-UPDRS I “Hallucinations and psychosis” item; semi-structured interview of the patient and/or in combination with Scale for the assessment of positive symptoms (SAPS); and The Positive and Negative Syndrome Scale (PANSS).

Preferably, a negative prognosis of the neuropsychological and/or neuropsychiatric status comprises a higher risk of developing, and/or worsening of, a dysfunction and/or an alteration of neuropsychological and/or neuropsychiatric parameters.

More preferably, dysfunction and/or alteration of neuropsychological parameters comprise at least one of cognitive decline, impairments or deficits in brain frontal-subcortical functions, impairments or deficits in brain posterior-cortical functions and PD dementia.

Advantageously, the method may further comprise a step of obtaining data regarding the neuropsychological status of the subject at a time T₀.

Advantageously, electrophysiological data of the subject are obtained through electroencephalography and/or electroencephalography (EEG) recordings.

Advantageously, a reference of frontal theta oscillatory frequency band power and/or activity refers to EEG recordings data obtained from at least one healthy volunteer or a population thereof, a subject with no report or presence of Minor Hallucination (MH) events or a population thereof, a subject with no dysfunction and/or alteration of neuropsychological parameters or a population thereof, and a subject diagnosed with a neurological or neuropsychiatric disease or a population thereof.

Advantageously, an enhanced frontal theta oscillatory frequency band power and/or activity compared to a reference is present in the absence of alterations in alpha and/or beta and/or gamma oscillatory pattern(s).

Advantageously, an enhanced frontal theta oscillatory frequency band power and/or activity compared to a reference is present in the absence of report or presence of Visual Hallucination (VH) events.

Advantageously, an enhanced frontal theta oscillatory frequency band power and/or activity compared to a reference is detected only in frontal-subcortical brain regions of a subject.

Advantageously, the method may further comprise a step of obtaining data regarding the neuropsychological and/or neuropsychiatric status of the subject at a time Tᵢ comprised between 1 month and 20 years from T₀.

Advantageously, the method is computer-implemented.

Another object of the invention relates to a data processing apparatus comprising a processor configured to perform the method of the invention.

Still another object of the invention relates to computer program comprising instructions which, when the program is executed by a processing apparatus, such as the system according to the invention, cause the processing apparatus to carry out the method of the invention.

Still another object of the invention relates to a computer-readable data carrier having stored thereon the computer program of the invention.

Still another object of the invention relates to database comprising:
i) data regarding the neuropsychiatric and/or neuropsychological status of subjects at a time T₀, said data comprising information related to Minor Hallucination (MH) events; and
ii) electrophysiological data of the subjects at a time T₀ comprising frontal theta oscillatory frequency band power and/or activity.

Advantageously, the database further comprises data regarding the neuropsychological status of the subjects at a time T₀.

Advantageously, the database further comprises at least one of:
i) data regarding the neuropsychological status of the subjects at a time Tᵢ, wherein Tᵢ is comprised between 1 month and 20 years from To.

Particularly, subjects are patients suffering from a neurological disease and/or neuropsychiatric disease selected from a list comprising Alzheimer's disease, Parkinson's disease (PD), Dementia with Lewy bodies (DLB), REM sleep behaviour disorder (RBD), major depression, schizophrenia, preferably Parkinson's disease (PD) patients.

Further embodiments of the present invention are defined by the appended claims.

The above and other objects, features and advantages of the herein presented subject-matter will become more apparent from a study of the following description with reference to the attached figures showing some preferred aspects of said subject-matter.

### BRIEF DESCRIPTION OF DRAWINGS AND TABLES

FIG. 1. Prevalence of MH in PD. The figure illustrates the sum of patients with a specific MH (left bar plot) among the PD-MH. The figure also illustrates the sum of patients experiencing only one MH (violet dots indicate which MH, top bar plot indicates the sum of patients experiencing the MH) as well as the co-occurrence of two MH (two or more violet dots linked by a black line indicate which MH are co-experienced, the top bar plot indicates the sum of patients experiencing those MH). The figure was generated with UpSetR (Conway et al., n.d.) an R package.

FIG. 2. Association between frontal-subcortical cognitive functions and frontal theta power in PD-MH. A. Reconstructed aperiodic-adjusted theta peaks, for PD-MH (violet) and PD-nMH (non-Minor Hallucination) (orange), thicker lines indicate the mean of each group. Thinner lines indicate the single patient data. B. Topography of F-values the interaction between patients' group and frontal-subcortical cognitive functions. White highlighted dots indicate electrodes showing a significant (p-values<0.05; FDR-corrected) interaction between the theta frequency band and frontal-subcortical cognitive functions. Note that 4 of 5 electrodes were over frontal scalp regions, bilaterally. C. Frontal theta oscillatory power is associated with frontal-subcortical cognitive functions in PD-MH. Higher power is associated with lower cognitive functions. Single dots represent the value for each patient (average of the electrodes showing a significant interaction between patients' group and frontal-subcortical cognitive functions). D. Frontal theta oscillatory power is not associated with frontal-subcortical cognitive functions in PD-nMH. Single dots represent the value for each patient. Significance was obtained with permutation tests and multiple comparisons were corrected with FDR.

FIG. 3. Association between frontal-subcortical cognitive functions and frontal central frequency in PD-MH. A. Reconstructed aperiodic-adjusted theta peaks, for PD-MH (violet) and PD-nMH (orange), thicker lines indicate the mean of each group. Thinner lines indicate the single patient data. B. Topography of F-values obtained from the interaction between patients' group and frontal-subcortical cognitive functions. White highlighted dots indicate electrodes showing a significant (p-values<0.05; FDR-corrected) interaction for the center frequency (4-13 Hz) and the frontal-subcortical cognitive functions. C. Center frequency is associated with frontal-subcortical cognitive functions in PD-MH. Lower center frequency is associated with lower cognitive functions. Single dots represent the value for each patient (average of the electrodes showing a significant interaction between patients' group and frontal-subcortical cognitive functions). D. Center frequency is not associated with frontal-subcortical cognitive functions in PD-nMH. Single dots represent the value for each patient. Significance was obtained with permutation tests and multiple comparisons were corrected with FDR.

FIG. 4. Longitudinal progression of the frontal-subcortical cognitive functions. Estimated marginal effects, by the linear mixed-model, for the five-year longitudinal PD-CRS follow-up data on the frontal-subcortical cognitive functions are shown. A. In PD-MH patients frontal-subcortical cognitive functions decline significantly (p-value<0.05) over 5 years. B. In PD-nMH patients frontal-subcortical cognitive functions do not decline significantly over 5 years. C. Difference in frontal-subcortical cognitive functions at the third assessment (year 5). PD-MH show a lower frontal-subcortical cognitive functions than PD-nMH. The bigger dots on the sides indicate the mean of the group. The error bars indicate 95% confidence interval. Asterisk indicates a statistical difference.

FIG. 5. Frontal theta power during the first assessment anticipates cognitive decline occurring over 5 years. A. Results of the linear regression show that in PD-MH patients frontal theta power, as measured during the first assessment, is associated with the frontal-subcortical cognitive decline (normalized decline, see methods), as measured during the third assessment (5 years later). B. Results of the linear regression show that in PD-nMH patients frontal theta power, as measured during the first assessment, is not significantly associated with the frontal-subcortical cognitive decline (normalized decline, see methods), as measured during the third assessment (5 years later). P-values were computed with permutation marginal tests for linear models.

FIG. 6. Illustration of a hypothetical EEG signal. A. Illustration of a EEG power signal as two distinguishable signals: the aperiodic (1/f) background signal (dashed line); and the periodic components (dark blue) The periodic signal is composed of the: i) central frequency; ii) adjusted power; and iii) bandwidth (not shown). Those features of the periodic signal can change across groups and/or experimental conditions. The aperiodic signal can vary in Offset (or intercept) (B) or in exponent (or slope) (C).

FIG. 7. Topographies showing aperiodic signals as a function of MH. A. Topographies indicate the exponent (aperiodic signal) for PD-MH (left) and PD-nMH (right), and the topography on the bottom indicates the t-values for the statistical difference. No statistical difference was observed between patient groups. B. Offset for an exemplary electrode (Cz). Single dots indicate the individual participant's exponent. The bigger dots on the sides indicate the mean of the group (PD-MH: violet; PD-nMH: orange). The error bars indicate 95% confidence interval. C. Topographies indicate the exponent (aperiodic signal) for PD-MH (left) and PD-nMH (right), and the topography on the bottom indicates the t-values for the statistical difference. No statistical difference was observed between patient groups. B. Offset for an exemplary electrode (Cz). Single dots indicate the individual participant's offset. The bigger dots on the sides indicate the mean of the group (PD-MH: violet; PD-nMH: orange). The error bars indicate 95% confidence interval.

FIG. 8. Longitudinal follow-up of the frontal-subcortical cognitive functions. Raw data for the five-year longitudinal PD-CRS follow-up data on the frontal-subcortical cognitive functions are shown. Single dots indicate an individual patient's cognitive score. The bigger dots on the sides indicate the mean of the group. The error bars indicate 95% confidence interval. Asterisk indicates a statistical difference.

FIG. 9. Schematic representation of a system according to an embodiment of the invention for predicting a prognosis of a neuropsychological and/or a neuropsychiatric status in a subject.

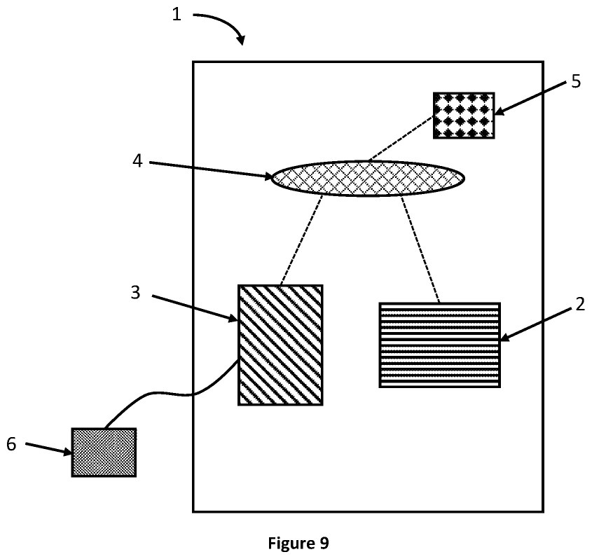

FIG. 10. Exemplary flowchart representation of the method steps according to an embodiment of the invention.

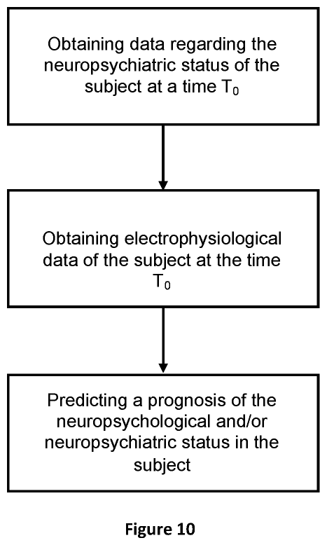

FIG. 11. Exemplary flowchart representation of the method steps according to an embodiment of the invention.

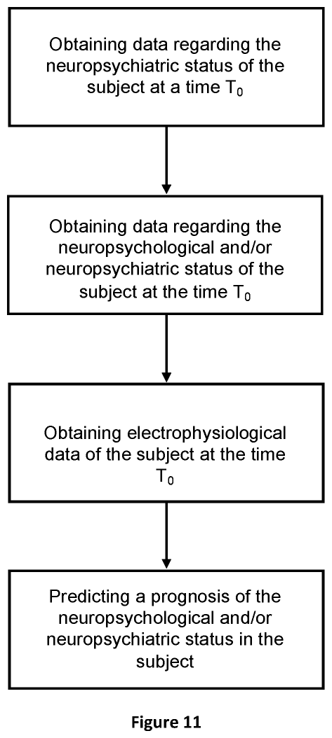

FIG. 12. Exemplary flowchart representation of the method steps according to an embodiment of the invention.

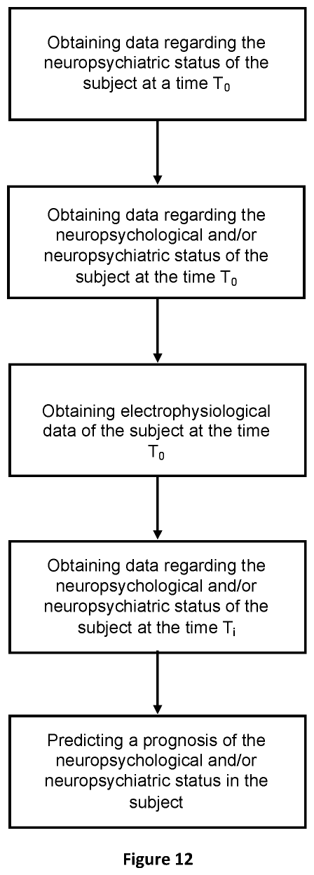

FIG. 13. Schematic representation of a robotic master-slave system for inducing a Presence Hallucination (PH) such as a FoP hallucination according to an embodiment of the invention.

Table 1. Clinical and demographic variables for PD-MH and PD-nMH. Appendix a indicates Welch test, b Chi-squared, and c indicates U Mann Whitney test.

Table 2. Statistical results of the models investigating modulations of the oscillatory power as a function of MH and frontal-subcortical cognitive functions (interaction between the two variables). Effect indicate which frequency is considered in the model. Permutation p-values are reported after FDR correction for multiple comparison.

Table 3. Statistical results of the models investigating modulations of the oscillatory power as a function of MH and posterior cognitive functions (interaction between the two variables). Effect indicate which frequency is considered in the model. Permutation p-values are reported after FDR correction for multiple comparison.

Table 4. Statistical results of the models investigating modulations of the oscillatory power as a function of MH and UPDRS-part 3 (interaction between the two variables). Effect indicate which frequency is considered in the model. Permutation p-values are reported after FDR correction for multiple comparison.

Table 5. Statistical results of the models investigating modulations of the center frequency in the theta-alpha (4-13 Hz) frequency band, as a function of MH and frontal-subcortical and posterior cognitive functions (interaction between the two variables; one model per cognitive functions). Effect indicate which cognitive function is considered in the model. Permutation p-values are reported after FDR correction for multiple comparison.

Table 6. Statistical results of the models investigating modulations of the oscillatory power for several frequency bands, as a function of MH. Statistical models were applied to each electrode independently. Permutation p-values are reported after FDR correction for multiple comparison. Results show that no statistical significant difference in oscillatory power is associated to MH.

Table 7. Statistical results of the models investigating modulations of the center frequency in the theta-alpha (4-13 Hz) frequency range, as a function of MH. Statistical models were applied to each electrode independently. Permutation p-values are reported after FDR correction for multiple comparison. Results show that PD-MH have a lower center frequency on central electrodes. Asterisks indicate significant effects.

Table 8. Statistical results of the models investigating modulations of the aperiodic signals (Offset and Exponent) as a function of MH and frontal-subcortical cognitive functions (interaction between the two variables). Effect indicates which between offset and slope are considered in the model. Permutation p-values (indicating the interaction term) are reported after FDR correction for multiple comparison.

### DETAILED DESCRIPTION OF THE INVENTION

The subject-matter described in the following will be clarified by means of a description of those aspects which are depicted in the drawings. It is however to be understood that the scope of protection of the invention is not limited to those aspects described in the following and depicted in the drawings; to the contrary, the scope of protection of the invention is defined by the claims. Moreover, it is to be understood that the specific conditions or parameters described and/or shown in the following are not limiting of the scope of protection of the invention, and that the terminology used herein is for the purpose of describing particular aspects by way of example only and is not intended to be limiting.

Unless otherwise defined, technical and scientific terms used herein have the same meaning as commonly understood by one of ordinary skill in the art to which this invention belongs. Further, unless otherwise required by the context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated. Further, for the sake of clarity, the use of the term “about” is herein intended to encompass a variation of +/−10% of a given value.

Non-limiting aspects of the subject-matter of the present disclosure will be described by way of example with reference to the accompanying figures, which are schematic and are not intended to be drawn to scale. For purposes of clarity, not every component is labelled in every figure, nor is every component of each aspect of the invention shown where illustration is not necessary to allow those of ordinary skill in the art to understand the invention.

As used in the following and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where for the description of various embodiments use is made of the term “comprising”, those skilled in the art will understand that in some specific instances, an embodiment can be alternatively described using language “consisting essentially of” or “consisting of.”

The present invention is based, at least in part, on the inventor's hypothesis that a model-driven EEG approach to measure periodic and aperiodic properties of resting-state EEG data, combined with in-depth neuropsychiatric interviews to investigate hallucinations, would be valuable for conveniently investigating whether MH in adult or elderly subjects, and particularly PD patients, are associated with specific alterations in oscillatory brain activity, and whether such changes are associated with specific neuropsychological deficits and more rapid cognitive decline, indicating for instance a more severe form of PD. In fact, minor hallucinations in Parkinson's disease are frequent non-motor symptoms in PD patients, and growing evidence suggest that they anticipate complex visual hallucinations, and therefore possible PD-associated psychosis. However, whether MH anticipate also cognitive decline and its severity, is actually unknown. Advantageously, neuropsychological examinations to determine cognitive functions, at an early and/or later assessment stage, may be associated to the above-mentioned neuropsychiatric and electrophysiological status assessment to have a more complete view and facilitate a longitudinal follow-up of the cognitive subject's parameters.

In a first aspect, the invention relates to a system 1 for predicting a prognosis of a neuropsychological and/or a neuropsychiatric status in a subject.

The system 1 may comprise a first device 2 (or a first measuring device 2 or a first input device 2) for measuring or obtaining data regarding the neuropsychiatric and/or neuropsychological status of a subject, for example at a time T₀.

Said data may comprise information related to Minor Hallucination (MH) events.

The first device 2 may comprise a terminal for a subject or an operator to enter neuropsychiatric and/or neuropsychological status. For example, questions regarding the neuropsychiatric and/or neuropsychological status may be displayed on a screen to gather information or data regarding the subject neuropsychiatric and/or neuropsychological status. The first device may gather information regarding the subject neuropsychiatric and/or neuropsychological status and process the information to determine the neuropsychiatric and/or neuropsychological status of the subject, in particular status related to Minor Hallucination (MH).

Information related to Minor Hallucination (MH) events may be obtained through at least one of MDS-UPDRS I “Hallucinations and psychosis” item; semi-structured interview of the patient and/or in combination with Scale for the assessment of positive symptoms (SAPS); and The Positive and Negative Syndrome Scale (PANSS).

The first device 2 may be connected to a memory or to a database comprising a subject neuropsychiatric and/or neuropsychological status.

The first device 2 may measure or obtain data regarding the neuropsychological status of the subject, for example at a given time or at a time T₀.

In a particular embodiment, the first device 2 is a master-slave robotic system configured to induce specific altered states of bodily consciousness, particularly Presence Hallucination (PH) such as a FoP hallucination, by manipulating sensorimotor inputs, such as the robotic system disclosed in U.S. Patent Publication No. 2016/0176053, the entire contents thereof being herewith incorporated by reference.

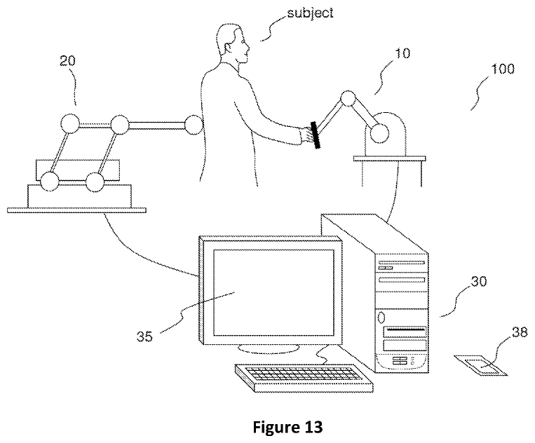

As shown in FIG. 13, the master-slave robotic system 100 may comprise a master device 10 (the “master”) having unidirectional control over one or more other slave devices 20 (the “slave(s)”). Both devices may be governed by software that is executed on a computer device 30, and they are operably connected among them in order to reproduce specific subject's induced movement and the related feedback.

A subject S may be connected with the robotic master device 10 so that (s)he can move, move on or manipulate it. When the subject moves, moves on or manipulates the master device 10 through moving parts of his/her body, preferably through limbs or extremities (in the variant shown his/her hand), (s)he is directly or indirectly touched by the slave device 20 according with a movement of master device 10 movement, preferably in a non-limb part of the body such as for instance the trunk. The term “according” means in a proper or appropriate way, i.e. in a way that suits the facts, needs, or requirements of a situation.

Particularly, the movement of the slave device 20 can perfectly mirror the movement of the master device 10 both in terms of spatial and temporal coordinates, or those movements can be performed in e.g. an asynchronous and/or asymmetric fashion. The mismatch (temporal and/or spatial and/or force mismatch) introduced by the robotic system is such that the subject receives spatially and/or temporally and/or force conflicting sensorimotor stimulation(s) up to a “break point” of sensory alteration in which the illusion is reached. One embodiment of the robotic system 100 is composed of or comprise a commercial master haptic interface, the Phantom Omni (SensAble Technologies), and a three degree-of-freedom (DOF) slave robot.

In one embodiment, a temporal mismatch may be introduced by the robotic system, that is, the conflicting sensorimotor stimulation is given by an asynchronous response of the slave device 20 compared to the activation of the master device 10 driven by the operating subject, so that the subject is touched by the slave device with a short delay, usually between 50 ms and 500 ms, compared to the induced movement of the master device. It has been in fact shown in the past by the present inventors that during an asynchronous stimulation, i.e. a stimulation by which the touch on the subject's body provided by the slave device is temporary delayed vis-à-vis the corresponding master device's movement, a subject is able to experience a Presence Hallucination (PH) such as a FoP. However, the use of the robotic system is not limited to temporal mismatches, and can be envisaged to induce force and/or spatial mismatches, alone or in combination to a temporal mismatch.

In one embodiment, the robotic system may be operably connected with a computer device 30 executing a computer program, the computer program comprising instructions to modulate the time and/or space and/or force activation of the slave device in response to the activation of the master device, to record data regarding the difference in time and/or space and/or force activation.

In embodiments, it is possible to compare the recorded data with reference data and to graphically or numerically, or otherwise display information with a display device 35, to show the result of the comparison on a display device 35, in order to extrapolate for instance proneness of a subject to experience a Presence Hallucination (PH) such as a FoP hallucination.

Also, according to one aspect of the present invention, a non-transitory computer readable medium 38 may be provided, the computer readable medium 38 having computer instructions recorded thereon for performing a method when executed by a computer device 30 having a processor that is in operative connection with a robot master device 10 and a robotic slave device 20 for interaction with the subject S.

Through the robotic system according to this embodiment, it is possible to record data concerning the difference in time and/or space and/or force activation of the slave device in response to the activation of the master device, such as for example the temporal mismatch in terms of master device activation/slave device response delay, and possibly compare the results of the recorded data with a set of reference data. The data can be a set of reference sensorimotor data and can have been obtained from e.g. healthy subjects upon administration of a spatially and/or temporally and/or force conflicting sensorimotor stimulation(s) thereon.

Without being bound to any theory, the recorded data and/or the results of comparisons with reference data can be positively or negatively associated with a neuropsychological or more generally neurological status of a subject, such as the likelihood to experience Presence Hallucination (PH) such as a FoP, at the moment of the test or in the future.

In embodiments, the conflicting stimulation, such as the above-described temporal mismatch-master device activation/slave device response delay—may be modulated (e.g., increased) gradually or sharply over time by the computer device until a so-called “break point.” Once the break point is reached, specific mismatches can be associated to it, allowing to generate different sub-populations, according to those parameters. In one embodiment, for each specific temporal mismatch, for example a delay between the master and the slave, a subject is asked to perform a certain number of touches, i.e. moving the master device in front and receiving touch on the back by the slave device, such as between five (5) to ten (10) touches, after which is asked to report whether he had the feeling of a presence. This can be done by a forced-choice task: “did you feel somebody behind you, touching you?” This is reported in a graph as proportion, or probability, of reporting a Presence Hallucination (PH) such as a FoP for a given specific delay over a certain number of trials/repetitions for delay. The delay can be modulated in the various test repetitions, so that the test is repeated over time with different conflicting sensorimotor parameters (e.g. time delays between the master and the slave devices).

The system 1 may further comprise a second measuring device 3 for measuring electrophysiological data of the subjects, for example at a time T₀.

The electrophysiological data may comprise frontal theta oscillatory frequency band power and/or activity.

The second measuring device 3 may comprise electroencephalogram (EEG) device 6 to measure electrophysiological data of the subject.

The system 1 may comprise a data analyzing device 4 for gathering and analyzing the data regarding the neuropsychiatric and/or neuropsychological status and the electrophysiological data and comparing the data to a reference.

Following the gathering and analyzing of the data, a report or a presence of Minor Hallucination (MH) events, together with an enhanced frontal theta oscillatory frequency band power and/or activity is indicative of a negative prognosis of the neuropsychological and/or neuropsychiatric status in the subject.

The prognosis may be given to an operator or the subject for example by a visualisation device and/or stored in a memory or a database.

The system 1 or the data analyzing device 4 may also comprise a calculation means or a processor, for example an embedded computational system and/or conventional computer connected to the first device 2 and/or to the second measuring device 3 to capture or obtain data from the first device 2 and/or to the second measuring device 3 and/or from a database.

The data analyzing device 4 (or the calculation means or the processor) may also be connected to the other elements of the system and configured to control and command these elements to permit operation of the system 1.

The system 1 may include a memory 5 (for example, semiconductor memory, HDD, or flash memory) configured to store or storing at least one program or processor executable instructions. The at least one program or processor executable instructions may comprise instructions permitting, for example, to control and command the first device 2, the second measuring device 3, the data analyzing device 4, the memory 5 and the other system elements. The processor executable instructions may comprise instructions permitting to obtain/receive and process the data obtained from the first device 2 or the second measuring device 3.

The analyzing device 4 (or the processor) and the memory can be, for example, included in a computer, portable laptop or a portable device such a s a smart phone or device. The program or processor executable instructions can be provided, for example, as custom Matlab functions, Phyton, C++ and/or VHDL.

The processor executable instructions can include instructions permitting various different actions concerning capturing and processing data of the present disclosure.

The processor executable instructions are provided or obtained by the processer for execution.

The first device 2, the data analyzing device 4 and/or the second measuring device 3 and/or the memory 5 and/or the visualisation device may be in operative connection permitting various different actions of the system 1 concerning capturing and processing data of the present disclosure.

In another aspect, the invention relates to a method for predicting a prognosis of the neuropsychological and/or neuropsychiatric status in a subject, the method comprising the steps of at least:
i) obtaining data regarding the neuropsychiatric status of the subject at a time T₀, said data comprising information related to Minor Hallucination (MH) events; and
ii) obtaining electrophysiological data of the subject at a time T₀ comprising frontal theta oscillatory frequency band power and/or activity,
wherein the report or the presence of Minor Hallucination (MH) events, together with an enhanced frontal theta oscillatory frequency band power and/or activity compared to a reference, is indicative of a negative prognosis of the neuropsychological and/or neuropsychiatric status in the subject.

According the invention, for “time T₀” it is herein meant a point in time or a lapse of time spanning from one day up to 6 months, and time T₀ starts at the end of this time or time lapse of the last of the step to be performed according to the method of the invention.

As a way of example, time T₀ can be the moment in time when electrophysiological data of the subject are obtained or measured, or when data regarding the neuropsychiatric status of the subject are obtained or measured for example by the system according to the invention. Additionally, time T₀ can be considered as a time lapse of up to 6 months between obtaining electrophysiological data of the subject and obtaining data regarding the neuropsychiatric status of the subject, or vice versa.

The system and method according to the present invention is particularly suited to predict the prognosis and development of certain aspects of the neuropsychological and/or neuropsychiatric status of neurological or neuropsychiatric patients, such as patients suffering from one or more of a non-limiting list of disorders including Alzheimer's disease, Parkinson's disease (PD), Dementia with Lewy bodies (DLB), REM sleep behaviour disorder (RBD), major depression and schizophrenia, with a particular focus on PD patients and their dementia-like symptoms. However, the system and method of the invention is also perfectly apt and applicable to assess or infer neuropsychological and/or neuropsychiatric development, such as degeneration, of subjects not affected by, or not diagnosed with, neurological or neuropsychiatric disorders, such as elderly experiencing or having experienced Minor Hallucinations.

The term “prognosis”, as used herein, relates to the prediction of the likely or expected development of a disease, including whether the signs and symptoms will improve or worsen (and how quickly) or remain stable over time; expectations of quality of life, such as the ability to carry out daily activities; the potential for complications and associated health issues; and the likelihood of survival (including life expectancy). A prognosis is made on the basis of the normal course of the diagnosed disease, the individual's physical and mental condition, the available treatments, and additional factors. In the frame of the present invention, the term may refer to the prediction of the likelihood of a progression or degradation of symptoms of a neurological or neuropsychiatric disorders. The system and the predictive methods of the present invention can be used clinically to make treatment decisions by choosing the most appropriate treatment modalities for any particular patient.

The expression “negative prognosis” refers to a non-favourable outcome of a disease or non-recovery from a disease, such as a decreased patient survival and/or an early disease progression and/or increased or rapid neurological or neuropsychiatric disease degradation.

According to the invention, a negative prognosis of the neuropsychological and/or neuropsychiatric status comprises a higher risk of developing, and/or worsening of, a dysfunction and/or an alteration of neuropsychological and/or neuropsychiatric parameters in a subject. Dysfunction and/or alteration of neuropsychological parameters comprise at least one of cognitive decline, impairments or deficits in brain frontal-subcortical functions, impairments or deficits in brain posterior-cortical functions and PD dementia.

A “neuropsychiatric disease” or disorder, is a behavioral or mental pattern that causes significant distress or impairment of personal functioning. Such features may be persistent, relapsing and remitting, or occur as a single episode. Many disorders have been described, with signs and symptoms that vary widely between specific disorders.

A “hallucination” is a perception in the absence of external stimulus that has qualities of real perception. Hallucinations are vivid, substantial, and are perceived to be located in external objective space. They are distinguishable from several related phenomena, such as dreaming, which does not involve wakefulness; pseudohallucination, which does not mimic real perception, and is accurately perceived as unreal; illusion, which involves distorted or misinterpreted real perception; and imagery, which does not mimic real perception and is under voluntary control. Hallucinations can occur in any sensory modality-visual, auditory, olfactory, gustatory, tactile, proprioceptive. In the frame of the present invention, “Minor Hallucination” (MH) events include sense of presence, passage hallucinations and/or visual illusions such as pareidolias.

According to the invention, information related to Minor Hallucination (MH) events are preferably obtained from a subject through at least one of MDS-UPDRS I “Hallucinations and psychosis” item; semi-structured interview of the patient and/or in combination with Scale for the assessment of positive symptoms (SAPS); and The Positive and Negative Syndrome Scale (PANSS). However, such information can be obtained as well through ad hoc questionnaires, that is, questionnaires not officially validated by the clinical community. As a way of example, presence and type of minor hallucinations can be assessed using the Hallucinations and Psychosis item of the MDS-UPDRS Part I and a semi structured interview covering the types of minor hallucinations described in the literature. Generally speaking, and without limitation, participants with a sense of presence, passage hallucinations, visual illusions, and/or pareidolias at least monthly or at least once during the last three months before the test are categorized as minor hallucinators. Cognition can be assessed by e.g. the Parkinson's Disease-Cognitive Rating Scale (PD-CRS) in PD patients. To assess sleep disturbances and diurnal somnolence, the Rapid Eye Movement Sleep Behavior Disorder Screening Questionnaire and the Epworth Sleepiness Scale can be used, respectively, as well as overnight polysomnography (PSG) analysis.

In embodiments, the system according to the invention may be configured to obtain data regarding the neuropsychological status of the subject, for example at time T₀.

The method according to the invention may further comprise a step of obtaining data regarding the neuropsychological status of the subject at a time T₀.

Neuropsychological tests are specifically designed tasks used to measure a psychological function known to be linked to a particular brain structure or pathway. Tests are used for research into brain function and in a clinical setting for the diagnosis of deficits. They usually involve the systematic administration of clearly defined procedures in a formal environment. Neuropsychological tests are typically administered to a single person working with an examiner in a quiet office environment, free from distractions. As such, it can be argued that neuropsychological tests at times offer an estimate of a person's peak level of cognitive performance. Neuropsychological tests are a core component of the process of conducting neuropsychological assessment, along with personal, interpersonal and contextual factors. Most neuropsychological tests in current use are based on traditional psychometric theory. In this model, a person's raw score on a test is compared to a large general population normative sample, which should ideally be drawn from a comparable population to the person being examined. Normative studies frequently provide data stratified by age, level of education, and/or ethnicity, where such factors have been shown by research to affect performance on a particular test. This allows for a person's performance to be compared to a suitable control group, and thus provide a fair assessment of their current cognitive function. A person skilled in the art would readily figure out the most appropriate test(s) to evaluate the neuropsychological performances of a subject depending on the needs and circumstances, such as without limitation the Parkinson's Disease-Cognitive Rating Scale (PD-CRS) and/or tests listed in Baerresen K. M. et al., Neurodegener Dis Manag. 2015; 5 (3):191-201, or in Delis D. C., Neuropsychological Testing, Encyclopedia of Neuroscience, Academic Press, 2009, Pages 983-991, incorporated herein by reference.

According to an advantageous embodiment, the method of the invention may further comprise a step of obtaining data regarding the neuropsychological and/or neuropsychiatric status of the subject at a time Tᵢ comprised between 1 month and 20 years from To. For instance, in order to assess cognitive changes due to e.g. a disease and the presence of MH, cognitive functions can be screened through e.g. PD-CRS 24 and 60 months after the initial visit (T₀). During these follow-up visits no EEG are needed and can therefore not be recorded.

The method of the invention foresees a step of obtaining electrophysiological data of the subject at a time T₀ comprising frontal theta oscillatory frequency band power and/or activity, typically through electroencephalography (EEG) and/or electroencephalography (EEG) recordings.

In the frame of the invention, frontal theta oscillatory frequency band power and/or activity in a test subject is compared to a reference, and an enhanced frontal theta oscillatory frequency band power and/or activity in the subject is indicative of a negative prognosis of the neuropsychological and/or neuropsychiatric status in the subject.

A reference of frontal theta oscillatory frequency band power and/or activity refers to EEG recordings data obtained from at least one healthy volunteer or a population thereof, a subject with no report or presence of Minor Hallucination (MH) events or a population thereof, a subject with no dysfunction and/or alteration of neuropsychological parameters or a population thereof, and a subject diagnosed with a neurological or neuropsychiatric disease or a population thereof.

In embodiments, an enhanced frontal theta oscillatory frequency band power and/or activity compared to a reference is present in the absence of alterations in alpha and/or beta and/or gamma oscillatory pattern(s). In additional or alternative embodiments, an enhanced frontal theta oscillatory frequency band power and/or activity compared to a reference is present in the absence of report or presence of Visual Hallucination (VH) events. In additional or alternative embodiments, an enhanced frontal theta oscillatory frequency band power and/or activity compared to a reference is detected only in frontal-subcortical brain regions of a subject.

As a way of example, resting-state EEG data can be collected using a multichannel (e.g. 19-channel) EEG system. Resting-state EEG data can be collected with eyes-open for a period lasting 5 minutes. Continuous EEG can be acquired at 250 Hz through a 19-channel Biosemi ActiveTwo AD-box referenced to the common mode sense (CMS; active electrode) and grounded to the driven right leg (DRL; passive electrode), which functions as a feedback loop driving the average potential across the electrode montage to the amplifier zero. Continuous EEG may also be acquired at 250 Hz from 19 standard scalp sites (Fp1/2, F3/4, C3/4, T3/4, T5/6, P3/4, O1/2, F7/8, Fz, Cz, Pz) using passive tin electrodes mounted in an elastic cap and referenced to the two mastoid leads. To monitor for eye movements and blinks, the horizontal and vertical electro-oculograms (EOG) are recorded with three external electrodes placed on the right and left temple (to measure horizontal eye movements) and below the right eye (to measure vertical eye movements). Two additional electrodes (CMS: Common Mode Sense and DRL: Driven Right Leg) are used as reference and ground. Additionally, or alternatively, vertical eye movements can be monitored using a bipolar montage with two electrodes linked together and placed below each eye referenced to a third electrode placed centrally above the eyes. Horizontal eye movements can be monitored using two electrodes placed on the external canthi of each eye. Electrode impedances are kept below 5 kOhm. The electrophysiological signals can be filtered with a bandpass of 0.1-35 Hz and digitized at a rate of 250 Hz. EEG acquisition can be done using tools available on the trade, such as the Brain Vision Recorder and BrainAMP system (Brain Products GmbH; Germany), according to the manufacturer instructions. Data may be analyzed off-line with e.g. the EEGLAB toolbox for MATLAB. After importing, data are low-pass-filtered at 45 Hz, and high-pass filter at 1 Hz with a conventional FIR filter. After removing the electrodes and epochs contaminated by artifacts, data are re-referenced to the average reference. The resting-state period is divided into e.g. 2 second epochs.

As a way of example, frequency analysis can be performed using Welch's estimation of power spectra or using the Fourier transform using Hanning as tapers on data segments with length of 2-second window.

Electrophysiological data in humans are characterized by a prominent 1/f power distribution, often referred to as “background noise”. This 1/f-like power distribution (also defined as aperiodic signal), captures the phenomenon whereby the power at low frequencies is relatively greater, conversely power is progressively lower at higher frequencies. This pattern results in overall negatively sloped power spectrum across a wide range of frequencies. Most commonly used methods and, therefore most of the research, used to analyze oscillatory power neglect the role of this aperiodic signal. This introduces a possible confound, in which the identified neural oscillations contain a superposition of periodic and aperiodic signals. To avoid this confound in analyses the inventors advantageously implemented a method to estimate both aperiodic and periodic signals.

In a non-limiting example, the open-source, Python-based Fitting Oscillations and One-Over-F (FOOOF) (version 1.0) toolbox can be used to estimate both the periodic and aperiodic signals. In a non-limiting example, the FOOOF algorithm is restricted to eight oscillatory peaks within the 2-45 Hertz range and constrained the peak width between 2-10 Hz to reduce the risk of over overfitting. In addition, a threshold (a peak was greater than the noise floor of at least 1 standard deviations above the residuals) is used to label the peak as a genuine neural oscillation. All the other parameters are used as indicated by default. More details on the procedure can be found in Donoghue T. et al., Nat Neurosci 23, 1655-1665 (2020), incorporated herein by reference. The model is applied to electrode or cluster of electrodes and patients, whenever applicable.

As a way of example, to investigate modulations of the signals as a function of MH and cognitive functions in PD patients, models are performed with the periodic (i.e. power and center) or aperiodic signals (slope and offset) as dependent variable, and with Group (PD-MH and PD-nMH) and cognitive functions (PD-CRS; either for the frontal-subcortical subscore or the posterior subscore) as covariates (updrs-3 scores were used instead of the cognitive functions of the control analyses). An interaction term between the two covariates is used in the model. Models are independently applied to electrodes or each cluster of electrodes and frequency band of interest (theta, alpha, beta, gamma). The significance may be estimated based with a permutation tests for ANOVA. Correction for multiple comparison for the number of electrodes may be obtained with False Discovery Rate (FDR), applied to each frequency independently. Patients for which a peak in a determined frequency is not observed/measured may be excluded from the statistical models for the given frequency band tested.

As a way of example, to investigate modulations of the signals as a function of mild cognitive impairment (MCI), models can be performed with the periodic or aperiodic signals as dependent variable as a function of Group (PD-MCI and PD-nMCI). Models are independently applied to electrodes or each cluster of electrodes and frequency band of interest (theta, alpha, beta, gamma). The significance may be estimated based with a permutation tests for ANOVA. Correction for multiple comparison for the number of clusters of electrodes may be obtained with False Discovery Rate (FDR), applied to each frequency independently.

According to an object of the invention, the method as described in the present specification may be computer-implemented. Accordingly, still another object of the invention relates to a data processing apparatus comprising a processor configured to perform the method of the invention.

Still another object of the invention relates to computer program comprising instructions which, when the program is executed by a processing apparatus, such as the system according to the invention, cause the processing apparatus to carry out the method of the invention.

Still another object of the invention relates to a computer-readable data carrier having stored thereon the computer program of the invention.

A data processing apparatus according to the invention can be part of a system that can comprise for instance an operatively connected EEG system and/or a non-transitory computer readable medium containing a set of instructions that, when executed by data processing apparatus of the system of the invention, cause said data processing apparatus to operate the system to perform a method according to the invention.

The data processing apparatus of the invention can comprise any suitable device such as computers, smartphones, tablets, voice-activated devices (i.e. smart speakers/voice assistants) and the like, depending on the needs and circumstances.

The data processing apparatus may advantageously comprise memory storing software modules that provide functionality when executed by the processor. The modules include an operating system that provides operating system functionality for the apparatus. The system, in embodiments that transmit and/or receive data from remote sources, may further include a communication device, such as a network interface card, to provide mobile wireless communication, such as Bluetooth, infrared, radio, Wi-Fi, cellular network, or other next-generation wireless-data network communication. In other embodiments, communication device provides a wired network connection, such as an Ethernet connection or a modem.

The instructions contained by the non-transitory computer readable medium may comprise, among others:
instructions for operating an EEG analysis system; and/or
instructions for storing, comparing and/or analysing 1) data regarding the neuropsychiatric status of one or more subjects, 2) electrophysiological data of one or more subjects and/or 3) data regarding the neuropsychological status of one or more subjects.

In this context, still another object of the invention relates to a database comprising:
i) data regarding the neuropsychiatric and/or neuropsychiatric status of subjects at a time T₀, said data comprising information related to Minor Hallucination (MH) events; and

ii) electrophysiological data of the subjects at a time T₀ comprising frontal theta oscillatory frequency band power and/or activity.

Advantageously, the database further comprises data regarding the neuropsychological status of the subjects at a time T₀.

Advantageously, the database further comprises at least one of:

i) data regarding the neuropsychological status of the subjects at a time Tᵢ, wherein Tᵢ is comprised between 1 month and 20 years from To.

Particularly, subjects are patients suffering from a neurological disease and/or neuropsychiatric disease selected from a list comprising Alzheimer's disease, Parkinson's disease (PD), Dementia with Lewy bodies (DLB), REM sleep behaviour disorder (RBD), major depression, schizophrenia, preferably Parkinson's disease (PD) patients.

### EXAMPLES

In an implemented, non-limiting exemplary experimental setting, in order to assess the association between MH, specific alterations in oscillatory brain and more rapid cognitive decline, the inventors enrolled 75 patients with PD, and conducted a continuous monitoring after two years (67 patients) and after 5 years (53 patients). In PD patients with MH, oscillatory alterations in the theta band over frontal regions were reported, which were MH-specific (absent in PD patients without MH) and further associated with lower cognitive frontal-subcortical functions (but not with posterior neuropsychological symptoms). The longitudinal neuropsychological follow-up at 2 and 5 years extends these specific findings and shows that cognitive decline is more rapid and severe in those patients with MH and with stronger frontal theta alterations, measured five years earlier.

When comparing 75 PD patients with and without MH, neither the neuropsychological nor the neuropsychiatric examination allowed the inventors to identify an association with cognitive dysfunction; yet by integrating EEG, psychiatric and neuropsychological examinations, it was observed that frontal oscillatory theta activity is predominant in patients with MH, and are associated with lower cognitive frontal-subcortical functions (not with posterior functions), formally demonstrating that enhanced frontal theta oscillations, when associated with MH, result in specific and rapid cognitive decline, and thereby highlighting the potential of this non-motor triad in the early identification of a more severe form of PD associated with cognitive decline and psychosis.

### Participants

Seventy-five individuals participated in the current study. All those fulfilling MDS new criteria for PD with minor hallucinations (PD-MH)—sense of presence, passage hallucinations, visual illusions and/or pareidolias (n=31)—and without any hallucinations (PD-nMH; n=44) were prospectively recruited from a sample of outpatients regularly attending to the Movement Disorders Clinic at Hospital de la Santa Creu i Sant Pau, Barcelona. Individuals were diagnosed with PD by a neurologist with expertise in movement disorders. Each individual was interviewed regarding disease onset, medication history, current medications, and dosage (levodopa daily dose and dopaminergic agonist-equivalent daily dose). Motor status and stage of illness were assessed by the MDS-UPDRS-III and Hoehn & Yahr scale. The two subgroups of patients were comparable in gender, age, disease duration, dopaminergic doses, dopaminergic agonists, motor impairment severity, sleep disturbances, disease stage (Hoehn & Yahr scale) and cognition.

Exclusion criteria were history of major psychiatric disorders, cerebrovascular disease, conditions known to impair mental status other than PD, and the presence of factors that prevented MRI scanning (e.g. claustrophobia, MRI incompatible prosthesis). Patients with focal abnormalities in MRI or non-compensated systemic diseases (i.e. diabetes, hypertension) were also excluded. In patients with motor fluctuations, cognition was examined during the “on” state. All participants were on stable doses of dopaminergic drugs during the 4 weeks before inclusion. Patients were included if the hallucinations remained stable during the 3 months before inclusion in the study. No participant had used or was using antipsychotic medication. All subjects had normal or corrected-to-normal vision. Informed consent to participate in the study was obtained from all participants according to the Declaration of Helsinki. The study was approved by the local ethics committee.

### EEG Cluster Selection

As a way of example, to address the multiple comparisons problem of independently testing each electrode, the analysis may be restricted to priori clusters of electrodes on the scalp. First, two parietal clusters of electrodes where created (P7,O1,O2,P8; P3,Pz,P4) over parietal-occipital cortex were selected based on prior research indicating that the dominant oscillation of the brain typically originates in parietal-occipital cortex. Second, a central electrodes cluster (C3,Cz,C4) where created over the somatosensory cortex, as well as two clusters for the fronto-temporal electrodes (F7, T7 and F8, T8). Third, because theta oscillations are commonly observed in frontal-midline electrodes in adults and in cognitive decline related to neurodegeneration two distinct electrodes cluster over the frontal regions (F3, Fz, F4 and Fp1, Fp2) were created based on previous research in adults that finds this region as the location of peak power of theta oscillations.

### Estimating Oscillatory Power and Aperiodic Signals

Frequency analysis was performed using Welch's estimation of power spectra (Welch, 1967), then averaged across epochs and electrodes within each cluster (see EEG cluster selection). The power spectrum was estimated with the Fourier transform using Hanning as tapers, in the frequency 1 Hz to 45 Hz, with a frequency resolution of 0.5 Hz, for each 2 seconds trial. The power spectrum was calculated for each trial and then averaged. Power spectrum estimation was computed for each electrode and participant independently.

### Progression of the Cognitive Functions

To assess changes in cognitive functions measured at baseline (Year 0), 24 months (Year 2) and 60 months (Year 5), frontal-subcortical and posterior cognitive functions were analyzed with linear mixed-effects ANOVA models. Two distinct models were performed for the two PD-CRS subscores. The models were performed with the cognitive scores and the measures time points as fixed effect (interaction between the two terms) and with random intercepts for each participant. The significance of fixed effects was estimated with a permutation test (5000 iterations).

### EEG Activity at Baseline (Year 0) Anticipates Cognitive Decline (at Year 5)

Based on the results of the cognitive decline over the year, frontal-subcortical cognitive decline was quantified by calculating the difference in score between the two points (Year 0−Year 5) and normalized by dividing by the sum of the two scores (Year 0+Year 5). To investigate whether cognitive decline was associated with the theta power over the frontal electrodes (recorded at To) and was differently modulated between subgroups of patients, the inventors conducted a linear regression, and statistical significance was obtained with permutation test (5000 iterations), with theta (but also alpha, beta and gamma) power and subgroups as independent variables (and with an interaction term between the two), and cognitive decline as dependent variable.

### Minor Hallucinations (at First Evaluation, Semi-Structured Interview)

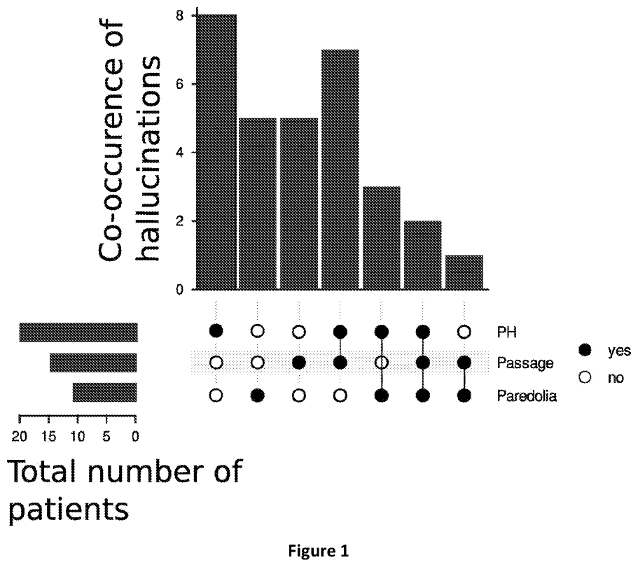

Based on the neuropsychiatric semi-structured interview, 75 patients with PD were grouped into those who reported MH (PD-MH; n=31) and those without MH (PD-nMH; n=44). In the PD-MH subgroup, in addition to MH, two patients also reported VH, one patient reported auditory hallucinations. Although the exact prevalence of MH in PD is still debated, evidence from previous studies suggests that approximately 50% of patients have MH, with presence and passage hallucinations as most the frequent MH (FIG. 1). The present results corroborate the prevalence of MH in PD, with 41.37% (31/75) of the patients experiencing MH.

### Demographical, Clinical, and Neuropsychological Data (at First Evaluation)

The demographic data did not show any significant (all p-values>0.05) differences in age or gender between the two patient subgroups (Table 1). MHs have been associated with the dosage of the dopaminergic medications. Yet, they have also been observed before the onset of motor symptoms and in the absence of any intake of dopaminergic medications, leaving the role of dopaminergic treatments in MH unresolved. The results show that the dosage of the dopaminergic treatments were not significantly (p-value=0.3 and p-value=0.9; for levodopa equivalent dose and dopamine agonists equivalent dose, respectively) different between patients that experience MH vs. those without, supporting the hypothesis that MH are linked to PD rather than (directly to) a medication side-effect.

The results of the neuropsychological examination did not reveal any significant differences between the PD-MH and PD-nMH patients. This was neither found for the frontal-subcortical cognitive functions (p-value=0.3) nor for the posterior functions (p-value=0.4). These results are in line with previous literature showing that, at the group level, PD patients with MH do not differ from those without MH for cognitive functioning. The inventors also analyzed whether the number of hallucinations (sum of different minor hallucinations experienced by a patient) was associated with the neuropsychological scores. Again, neither the frontal-subcortical cognitive score (rho=−0.14, p-value=0.46) nor the posterior cognitive score (rho=0.04, p-value=0.84) was associated with the number of MH.

Frontal Theta Oscillatory Power is Enhanced in Patients with MH and Associated with Frontal-Subcortical Cognitive Decline (at First Evaluation)

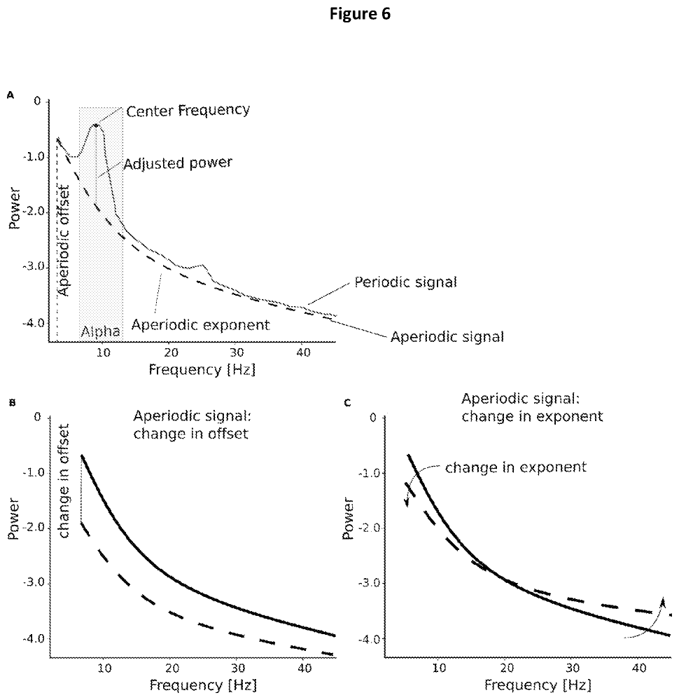

EEG signal is characterized by a background activity of the brain (1/f aperiodic signal; characterized by offset and slope) and genuine oscillatory signals (periodic signals; characterized by the center, power, and bandwidth of the peak; FIG. 6). To better understand and interpret EEG results it is critical to separate the aperiodic from periodic signals. In the present analysis of neural oscillations, the inventors focus on the power and center frequency of the dominant oscillation, and the analysis of the aperiodic signal included both the slope and offset.

### Periodic EEG Signal

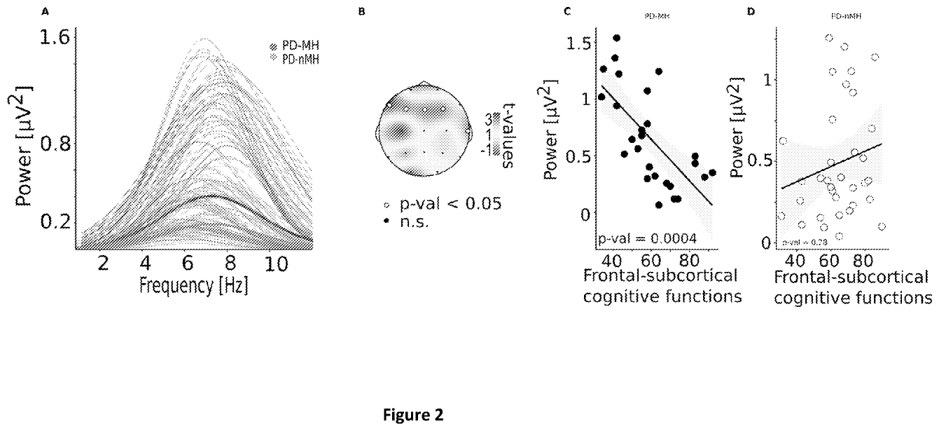

The inventors analyzed whether the oscillatory power is modulated by MH and whether the oscillatory power is associated with lower cognitive functions (frontal-subcortical and posterior PD-CRS). The results show that the oscillatory power within the theta frequency band (4-8 Hz; FIG. 2A-B) is significantly (p-values<0.05; FDR corrected) modulated by the frontal-subcortical cognitive functions and by MH (yes/no) (i.e. interaction between the two terms) (for PD-MH FIG. 2C; for PD-nMH FIG. 2D). Post-hoc analysis revealed that the association between theta power and frontal-subcortical cognitive functions was significant for PD-MH (p-value<0.001; FIG. 2C) but not for PD-nMH (p-value=0.28; FIG. 2D). Moreover, the present results indicate that for PD-MH theta oscillatory power was negatively associated with the frontal-subcortical score, with higher theta power associated with lower cognitive scores (FIG. 2C). The association between oscillatory activity and frontal-subcortical functions (as a function of MH: interaction MH and frontal-subcortical PD-CRS) was specific for the theta frequency band, as no other frequency band (alpha: 8-12 Hz, beta: 13-30 Hz, and gamma: 31-45 Hz) showed an association with frontal-subcortical cognitive functions (all interactions permutation p-values>0.05; FDR corrected; Table 2). In addition, this association was specific for electrodes over the frontal region, as no other significant (p-values>0.05; FDR corrected; Table 2) cluster of electrodes was observed for this interaction.

In addition, the results show that the frontal theta oscillatory power is modulated as a function of MH (p-values<0.05; FDR-corrected), with the theta power being enhanced in patients with MH (mean±SD: 0.81±0.45 μV2) when compared to patients without MH (mean±SD: 0.54±0.37 μV2). No other electrode or any of the other tested frequencies showed a significant modulation associated with MH (all p-value>0.05; FDR corrected).

The frontal theta pattern in PD-MH was only found for the frontal-subcortical functions, as there was no association between MH and the posterior cognitive functions in any of the tested frequencies and electrodes (all interactions p-values>0.05; FDR corrected; Table 3).

Because enhanced theta (and beta) power has been associated with motor impairment and its severity in PD patients and because the degree of motor impairment has been associated with cognitive decline, it could be argued that the present findings (i.e. enhanced theta oscillations associated with MH and frontal-subcortical cognitive decline) may reflect differences in motor symptoms. This was not the case (see Table 1). Additional control analysis, investigating whether changes in oscillatory activity are associated with MH and motor impairment (UPDRS-Part 3), did not show any significant frequency modulations in any brain region (for all interactions between MH and UPDRS-Part 3 the p-values>0.05; FDR corrected; Table 4) between groups as a function of patients' motor impairment. Moreover, PD-MH and PD-nMH did not show any difference in the UPDRS motor scores (Table 1).

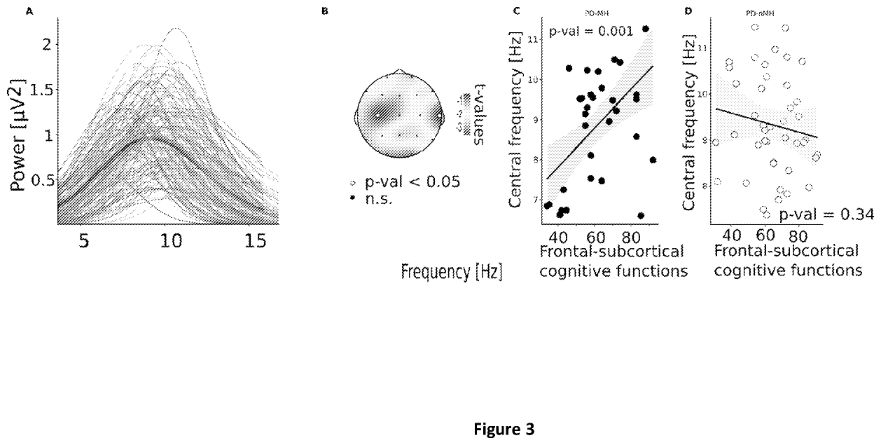

Previous research on cognitive decline in PD has described a frequency “shift” or slowing down from predominant alpha oscillations to predominant theta oscillations, after the onset of dementia. However, these interpretations were reached based on changes in power (intensity of the oscillation), rather than in the center frequency (the actual frequency—in Hz—at which the peak is observed). That is, the “shift” was inferred from heaving a reduced power in the alpha band and higher in the theta band, rather than a change in the frequency peak from 8-13 Hz (alpha) to 4-8 Hz (theta band). Therefore, the inventors tested whether the association between theta oscillations, MH and frontal-cognitive decline is also associated with a change in the center frequency (shift of the center frequency from alpha to theta). The inventors observed a significant (p-values<0.05; FDR corrected) modulation of the central frequency in the alpha-theta frequency ranges as a function of the interaction between the two terms (frontal-subcortical cognitive functions and by MH (yes/no)). This interaction was localized over central electrodes (see Table 5). Post-hoc analyses show that those central electrodes show a significant (p-value=0.001, FIG. 3B) association between the central frequency (in the alpha-theta frequency range) and the frontal subcortical cognitive functions for patients with MH. This associations shows that, a reduction of the central frequency (i.e. shifting from alpha to theta) is associated with lower cognitive functions. This was not the case for patients without MH (p-value=0.49, FIG. 3D). The inventors did not observe any significant interaction (all p-values>0.05; FDR corrected; Table 5). Additional analysis showed the absence of significant (all p-values>0.05; FDR corrected; Table 6) modulations of the center frequency for the posterior cognitive functions in the theta-alpha frequency range (interaction between the two terms (posterior functions and MH (yes/no)). Collectively, these results suggest that the association between MH and frontal-subcortical cognitive function is due to both a power and central frequency changes.

### Aperiodic Signal

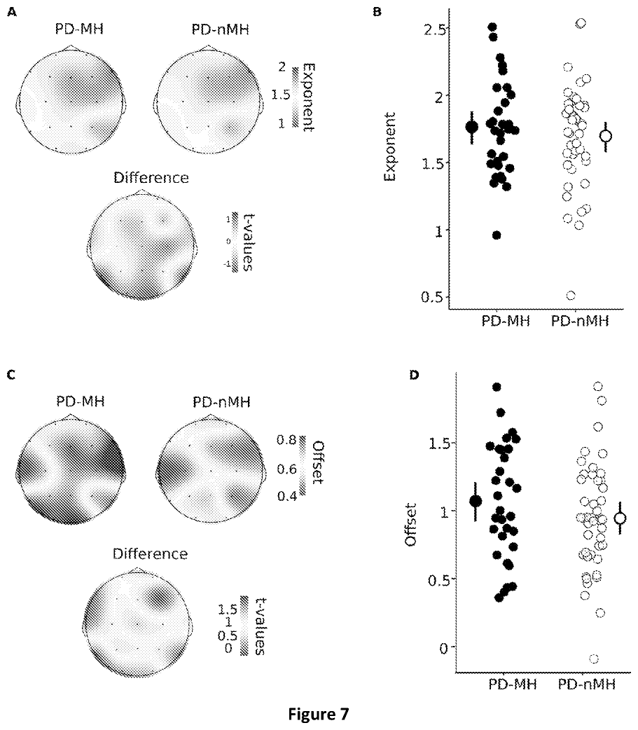

The precise neural mechanisms that alter the aperiodic slope of intrinsic neural activity remain an active area of research. However, a flattened slope of the aperiodic signal has been correlated with age and with age-related cognitive decline. Similarly, the offset of the aperiodic signal decreases with age. Therefore, the inventors investigated whether modulations of slope and/or offset might be related to changes as a function of MH (yes/no) and frontal-subcortical cognitive functions (interaction between the two terms). The results show that neither slope nor offset of the aperiodic signals are significantly modulated (p-values>0.05; Tables 7 and 8; FIG. 7) as a function of cognitive function (frontal-subcortical) and MH. These results demonstrate that the theta modulations the inventors observed are due to a change in oscillatory power and not to aperiodic modulations.

Cognitive Decline is More Severe in Patients with MH and Frontal-Theta Enhancement (2 and 5 Year Follow-Up Evaluations)

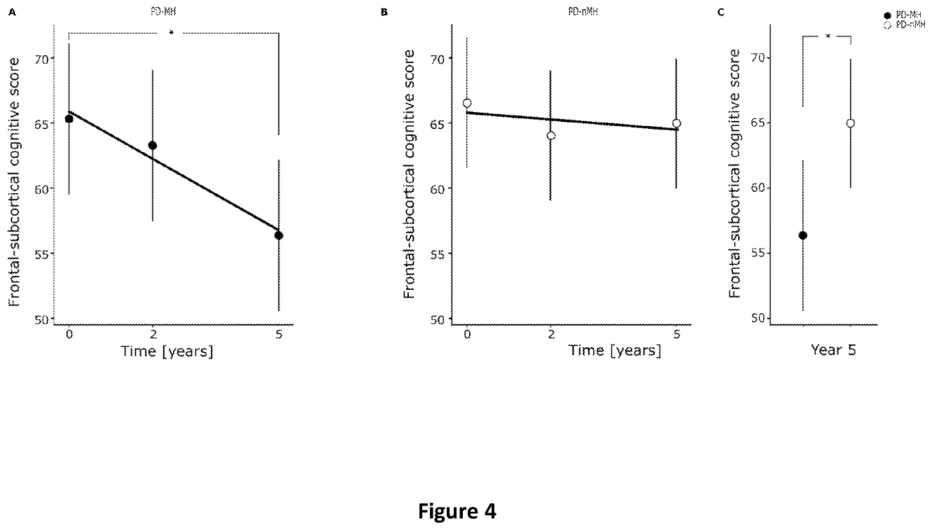

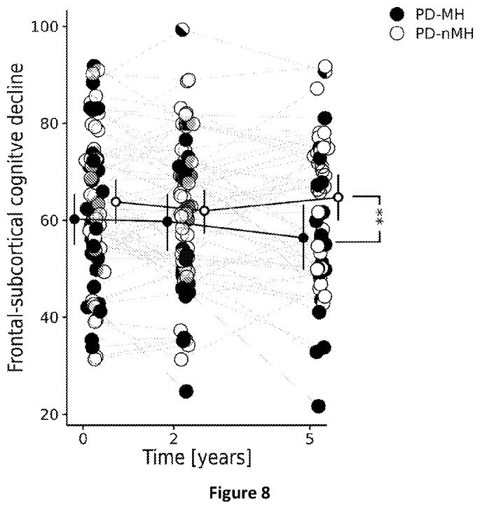

Neuropsychological follow-up at 2 years (n=67) and at 5 years (n=53) shows that the decline in frontal-subcortical cognitive functions was significantly more severe (p-value<0.01; interaction between MH and follow-up; FIGS. 4 and 8) in patients with MH versus patients without MH, especially after 5 years. This was not the case for the posterior cognitive functions decline (p-value=0.13).

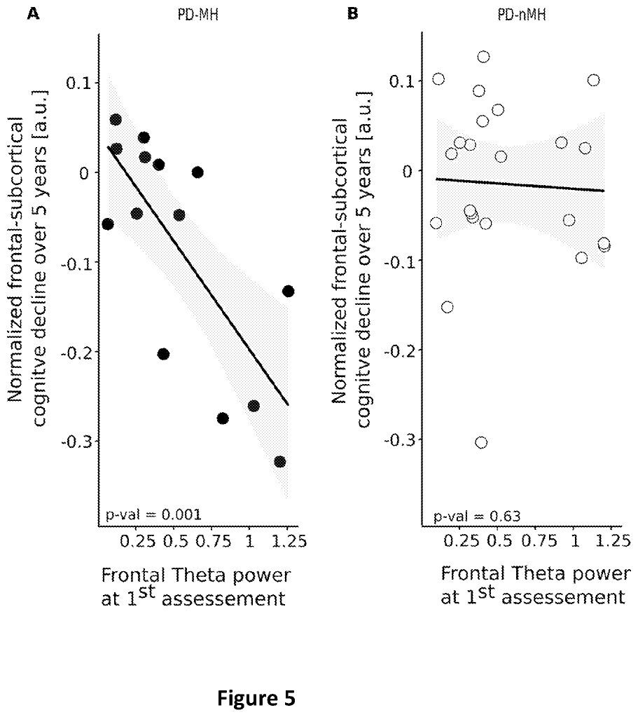

Informed by the results showing that frontal theta power is associated with frontal-subcortical cognitive score in patients with MH and the evidence that theta might be a predictor of cognitive decline, finally, the inventors investigated whether frontal theta power measured at the first evaluation anticipated frontal-cognitive decline measured 5 years later. Results show that for PD-MH patients higher frontal theta power during the first evaluation is associated with a more severe frontal-cognitive decline (p-value=0.001; FIG. 5A) over the 5 years. This was not the case for patients without MH (p-value=0.64; FIG. 5B; interaction between the two terms: p-value=0.01). None of the other frequencies showed such interaction (all p-value>0.05).

Conducting EEG, psychiatric and neuropsychological examinations in 75 patients with PD during a first evaluation, the inventors report an alteration selectively for frontal theta frequency band in PD patients with MH and show that these changes were associated with the severity of frontal-subcortical cognitive deficits. Enhanced frontal theta changes were not associated with posterior cognitive functions and were absent in PD patients without MH. When comparing the two groups of patients with and without MH, neither the neuropsychological nor the neuropsychiatric examination allowed the inventors to identify an association with cognitive dysfunction. However, by integrating the results of neuropsychiatric interviews with EEG (and with neuropsychological data), the inventors found that PD patients, specifically those suffering from MH, show enhanced theta oscillatory activity, in frontal brain regions, and that this enhanced oscillatory power is associated with lower cognitive scores in frontal-subcortical functions. Furthermore, 5 years longitudinal cognitive assessment show that patients with MH have a more rapid cognitive decline, and that the frontal enhanced theta power (measured during the first assessment) is associated with the severity of the cognitive decline.

Cognitive impairment is a very frequent non-motor symptom in PD, with its prevalence increasing as the disease progresses. The diagnosis of cognitive decline is usually based on the neuropsychological assessment of attention, working memory, language, memory, as well as executive and visuospatial functions. However, as comprehensive evaluations of cognitive function in patients with PD is not performed systematically and requires specialized staff, cognitive impairments are often detected when the symptoms are already at an advanced stage. Hallucinations might indicate different stages of the disease and cognitive decline, with VH associated with posterior (visuospatial) impairments, and are most often only detected at the middle-to-late stage of the disease, whereas MH are rather associated with frontal-subcortical dysfunctions and may already appear at earlier stages. Therefore, MH might be a particularly interesting early marker of a more server form of the disease. However, when comparing patients with and without MH, neuropsychological results alone do not allow to detect group differences, neither in frontal-subcortical cognitive functions nor in posterior visuospatial functions, making the prediction of which individual is at highest risk of severe and rapid cognitive decline particularly challenging. The present neuropsychological data are consistent with this observation by showing that patients with MH does not differ significantly from those without MH neither in the frontal nor in the posterior cognitive scores. However, the merging of neuropsychology, neuropsychiatric and oscillatory data, allowed the inventors to identify patients with more severe cognitive dysfunction. That is, for patients with MH, the severity of cognitive decline is associated with enhanced frontal theta. The longitudinal data (with a 5 years follow-up) further extend those results by showing that patients with MH have a more rapid cognitive decline compared to patients without MH, and that the frontal theta oscillatory power at the first assessment is associated with the severity of the cognitive decline.

Cognitive decline and PDD, independently from hallucinations, have previously been associated with enhanced theta oscillatory power (measured over the whole scalp), when comparing patients with vs. those without dementia (cross-sectional studies). This has also been reported for other neurodegenerative disorders characterized by cognitive decline. For instance, enhanced theta oscillations have been shown to correlate with the severity of cognitive fluctuations in dementia with Lewy bodies. The EEG results corroborate these findings, showing that enhanced theta measured over the whole scalp is associated with mild cognitive impairment MCI in the present PD patients.

Recent evidence shows that MH are not only the earliest hallucination in PD, but that they also share brain alterations with VH and are linked to early cognitive decline. Hallucinations in PD have previously been associated with enhanced theta oscillations. However, this was observed for VH, over the whole scalp, and was further associated with a concomitant reduction of higher frequencies. The present data show that, already during the first evaluation, when combined with psychiatric examinations (MH) and neuropsychological tests (frontal-subcortical functions), EEG changes reveal frontal theta changes, which are associated with frontal-subcortical cognitive decline, a marker for early cognitive impairment and is a predictor for mild cognitive impairment. The present study also shows that those modulations are not observed in any other brain region and in any other frequency band.

While the present results indicate that patients with MH are more likely to have more rapid cognitive decline (assessed by observing the evolution of the cognitive functions over 5 years), adding frontal theta will reveal which patient among those heaving MH is at higher risk. It has been suggested that enhanced theta activity in patents with cognitive decline reflects compensatory mechanisms for structural and functional changes observed in neurodegenerative disease with dementia. Thus, enhanced theta might reflect an early disruption of thalamocortical circuits or in medial frontal regions (i.e. altering working-memory and/or executive function). The contribution of theta oscillations to hallucinations has been linked to several potential mechanisms such as enhanced theta-bursts in fronto-thalamic regions, increased uncertainty in top-down activity resulting in incorrect sensory representations and hallucinations.

While an advanced stage of dementia (PDD) is usually associated with an enhanced theta activity measured over the entire brain (the results show that in patients with MH early frontal-subcortical cognitive decline is associated with more focal enhanced theta power over frontal regions (and not change in center frequency nor aperiodic signals). These changes did not affect posterior visuospatial functions and the present patients did not report VH (except for a very small number, i.e. 2/75), and did not arise from differences in clinical-demographic variables (including antiparkinsonian medication, motor impairment, and gender). Accordingly, the inventors speculate that in PD, enhanced theta starts in frontal-subcortical brain regions, associated with MH and frontal-subcortical cognitive symptoms, and, as the disease progresses, theta enhancement also propagates to posterior brain regions associated with VH and visuospatial cognitive impairment. Eventually, changes are characterized by a generalized theta enhancement and more prominent psychosis, characterized by MH and VH. In conclusion, these results suggest that PD patients at higher risk for a more severe form of the disease with cognitive decline and dementia can be identified by a triad of frontal theta, frontal-subcortical cognitive decline, and MH, not possible by using either of these measures alone or in other combinations.

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

### TABLES

TABLE 1

| | PD-MH | PD-nMH | |
|---|---|---|---|
| | (N = 31) | (N = 44) | p-values |
| Age (years) | 67.9 ± 7.31 | 66.1 ± 8.63 | 0.34^{a} |
| Gender (M/F) | 23/8 | 26/18 | 0.27^{b} |
| Disease duration (years) | 5.74 ± 2.22 | 4.77 ± 2.11 | 0.06^{a} |
| Education (years) | 12.7 ± 5.04 | 12.2 ± 4.68 | 0.7^{a} |
| Eq. dopamine agonists (mg) | 162 ± 137 | 153 ± 99.4 | 0.74^{a} |
| LEDD (mg) | 534 ± 250 | 464 ± 210 | 0.25^{a} |
| UPDRS-III ON | 26.5 ± 8.75 | 25.2 ± 7.30 | 0.49^{a} |
| PD-CRS Frontal-subcortical | 60.2 ± 15.4 | 63.8 ± 15.1 | 0.32^{a} |
| PD-CRS Posterior | 28.5 ± 1.95 | 28.2 ± 1.94 | 0.42^{a} |
| Hoehn & Yahr stage | 2.33 ± 0.38 | 2.18 ± 0.3 | 0.08^{c} |

Frontal theta oscillatory power is enhanced in patients with MH and associated with frontal-subcortical cognitive functions (interaction between the two terms).

TABLE 2

| | | | FREQ | PERMUTATION |
|---|---|---|---|---|
| CHAN | EFFECT | F-VALUES | BAND | P-VALUES (FDR-COR.) |
| FP1 | interaction | 0.05 | Theta | 0.87 |
| FP2 | interaction | 0.03 | Theta | 0.87 |
| F7 | interaction | 13.88 | Theta | 0.02* |
| F3 | interaction | 8.67 | Theta | 0.02* |
| FZ | interaction | 9.29 | Theta | 0.02* |
| F4 | interaction | 9.08 | Theta | 0.02 |
| F8 | interaction | 2.23 | Theta | 0.32 |
| T7 | interaction | 0.1 | Theta | 0.86 |
| C3 | interaction | 3.08 | Theta | 0.24 |
| CZ | interaction | 1.67 | Theta | 0.35 |
| C4 | interaction | 0.16 | Theta | 0.86 |
| T8 | interaction | 9.12 | Theta | 0.02* |
| P7 | interaction | 2.12 | Theta | 0.32 |
| P3 | interaction | 6.64 | Theta | 0.06 |
| PZ | interaction | 1.31 | Theta | 0.43 |
| P4 | interaction | 0.08 | Theta | 0.86 |
| P8 | interaction | 0.48 | Theta | 0.67 |
| O1 | interaction | 1.08 | Theta | 0.44 |
| O2 | interaction | 1.82 | Theta | 0.35 |
| FP1 | interaction | 0.3 | Alpha | 0.99 |
| FP2 | interaction | 0.45 | Alpha | 0.99 |
| F7 | interaction | 0 | Alpha | 0.99 |
| F3 | interaction | 0.12 | Alpha | 0.99 |
| FZ | interaction | 0.01 | Alpha | 0.99 |
| F4 | interaction | 0.02 | Alpha | 0.99 |
| F8 | interaction | 0.42 | Alpha | 0.99 |
| T7 | interaction | 0.5 | Alpha | 0.99 |
| C3 | interaction | 0.06 | Alpha | 0.99 |
| CZ | interaction | 0.33 | Alpha | 0.99 |
| C4 | interaction | 0.11 | Alpha | 0.99 |
| T8 | interaction | 0.28 | Alpha | 0.99 |
| P7 | interaction | 0 | Alpha | 0.99 |
| P3 | interaction | 0.17 | Alpha | 0.99 |
| PZ | interaction | 0.02 | Alpha | 0.99 |
| P4 | interaction | 0.23 | Alpha | 0.99 |
| P8 | interaction | 0.48 | Alpha | 0.99 |
| O1 | interaction | 0.09 | Alpha | 0.99 |
| O2 | interaction | 0.19 | Alpha | 0.99 |
| FP1 | interaction | 0.61 | Beta | 0.64 |
| FP2 | interaction | 0.92 | Beta | 0.64 |
| F7 | interaction | 0.65 | Beta | 0.64 |
| F3 | interaction | 1.29 | Beta | 0.64 |
| FZ | interaction | 0.02 | Beta | 0.9 |
| F4 | interaction | 0.05 | Beta | 0.86 |
| F8 | interaction | 0.09 | Beta | 0.86 |
| T7 | interaction | 0.63 | Beta | 0.64 |
| C3 | interaction | 0.28 | Beta | 0.75 |
| CZ | interaction | 2.76 | Beta | 0.64 |
| C4 | interaction | 2.25 | Beta | 0.64 |
| T8 | interaction | 0.14 | Beta | 0.85 |
| P7 | interaction | 0.37 | Beta | 0.75 |
| P3 | interaction | 1.6 | Beta | 0.64 |
| PZ | interaction | 1.82 | Beta | 0.64 |
| P4 | interaction | 0.88 | Beta | 0.64 |
| P8 | interaction | 0.63 | Beta | 0.64 |
| O1 | interaction | 2.42 | Beta | 0.64 |
| O2 | interaction | 0.86 | Beta | 0.64 |
| FP1 | interaction | 0.61 | Gamma | 0.53 |
| FP2 | interaction | 0.81 | Gamma | 0.53 |
| F7 | interaction | 4.12 | Gamma | 0.18 |
| F3 | interaction | 2.94 | Gamma | 0.2 |
| FZ | interaction | 1.5 | Gamma | 0.35 |
| F4 | interaction | 0.07 | Gamma | 0.82 |
| F8 | interaction | 5.42 | Gamma | 0.18 |
| T7 | interaction | 4.14 | Gamma | 0.18 |
| C3 | interaction | 0.05 | Gamma | 0.82 |
| CZ | interaction | 3.78 | Gamma | 0.18 |
| C4 | interaction | 3.37 | Gamma | 0.19 |
| T8 | interaction | 2.44 | Gamma | 0.23 |
| P7 | interaction | 0.74 | Gamma | 0.53 |
| P3 | interaction | 2.33 | Gamma | 0.23 |
| PZ | interaction | 3.15 | Gamma | 0.19 |
| P4 | interaction | 0.62 | Gamma | 0.53 |
| P8 | interaction | 0.05 | Gamma | 0.82 |
| O1 | interaction | 3.69 | Gamma | 0.18 |
| O2 | interaction | 6.28 | Gamma | 0.18 |

Oscillatory power is not modulated as a function of MH and posterior cognitive functions (interaction between the two terms

TABLE 3

| | | | FREQ | PERMUTATION |
|---|---|---|---|---|
| CHAN | EFFECT | F-VALUES | BAND | P-VALUES (FDR-COR.) |
| FP1 | interaction | 0.77 | Theta | 0.47 |
| FP2 | interaction | 0.12 | Theta | 0.77 |
| F7 | interaction | 6.44 | Theta | 0.1 |
| F3 | interaction | 4.78 | Theta | 0.13 |
| FZ | interaction | 6.26 | Theta | 0.1 |
| F4 | interaction | 6.47 | Theta | 0.1 |
| F8 | interaction | 3.38 | Theta | 0.17 |
| T7 | interaction | 0.35 | Theta | 0.63 |
| C3 | interaction | 0.06 | Theta | 0.82 |
| CZ | interaction | 2.95 | Theta | 0.19 |
| C4 | interaction | 1.83 | Theta | 0.31 |
| T8 | interaction | 4.23 | Theta | 0.15 |
| P7 | interaction | 2.81 | Theta | 0.19 |
| P3 | interaction | 0.84 | Theta | 0.47 |
| PZ | interaction | 1.21 | Theta | 0.39 |
| P4 | interaction | 0.45 | Theta | 0.6 |
| P8 | interaction | 1.76 | Theta | 0.31 |
| O1 | interaction | 3.39 | Theta | 0.17 |
| O2 | interaction | 5.94 | Theta | 0.1 |
| FP1 | interaction | 0 | Alpha | 0.99 |
| FP2 | interaction | 0.01 | Alpha | 0.99 |
| F7 | interaction | 0.17 | Alpha | 0.99 |
| F3 | interaction | 0.07 | Alpha | 0.99 |
| FZ | interaction | 0.6 | Alpha | 0.99 |
| F4 | interaction | 0 | Alpha | 0.99 |
| F8 | interaction | 1.66 | Alpha | 0.99 |
| T7 | interaction | 0.56 | Alpha | 0.99 |
| C3 | interaction | 1.13 | Alpha | 0.99 |
| CZ | interaction | 0.01 | Alpha | 0.99 |
| C4 | interaction | 0.71 | Alpha | 0.99 |
| T8 | interaction | 0.42 | Alpha | 0.99 |
| P7 | interaction | 0.9 | Alpha | 0.99 |
| P3 | interaction | 0 | Alpha | 0.99 |
| PZ | interaction | 0.08 | Alpha | 0.99 |
| P4 | interaction | 0.23 | Alpha | 0.99 |
| P8 | interaction | 0.69 | Alpha | 0.99 |
| O1 | interaction | 0.01 | Alpha | 0.99 |
| O2 | interaction | 0.11 | Alpha | 0.99 |
| FP1 | interaction | 0.09 | Beta | 0.94 |
| FP2 | interaction | 0.07 | Beta | 0.94 |
| F7 | interaction | 0.54 | Beta | 0.92 |
| F3 | interaction | 0.02 | Beta | 0.95 |
| FZ | interaction | 0.81 | Beta | 0.92 |
| F4 | interaction | 0.47 | Beta | 0.92 |
| F8 | interaction | 1.08 | Beta | 0.92 |
| T7 | interaction | 0.26 | Beta | 0.92 |
| C3 | interaction | 0.02 | Beta | 0.95 |
| CZ | interaction | 0.23 | Beta | 0.92 |
| C4 | interaction | 0.08 | Beta | 0.94 |
| T8 | interaction | 0.32 | Beta | 0.92 |
| P7 | interaction | 1.53 | Beta | 0.92 |
| P3 | interaction | 0.4 | Beta | 0.92 |
| PZ | interaction | 1.9 | Beta | 0.92 |
| P4 | interaction | 0.64 | Beta | 0.92 |
| P8 | interaction | 1.01 | Beta | 0.92 |
| O1 | interaction | 0 | Beta | 0.97 |
| O2 | interaction | 0.52 | Beta | 0.92 |
| FP1 | interaction | 0.02 | Gamma | 0.89 |
| FP2 | interaction | 0.24 | Gamma | 0.75 |
| F7 | interaction | 1.05 | Gamma | 0.63 |
| F3 | interaction | 1.4 | Gamma | 0.63 |
| FZ | interaction | 1.4 | Gamma | 0.63 |
| F4 | interaction | 0.29 | Gamma | 0.75 |
| F8 | interaction | 0.28 | Gamma | 0.75 |
| T7 | interaction | 1.31 | Gamma | 0.63 |
| C3 | interaction | 0.47 | Gamma | 0.75 |
| CZ | interaction | 0.02 | Gamma | 0.89 |
| C4 | interaction | 1.29 | Gamma | 0.63 |
| T8 | interaction | 0.88 | Gamma | 0.63 |
| P7 | interaction | 0.16 | Gamma | 0.75 |
| P3 | interaction | 0.96 | Gamma | 0.63 |
| PZ | interaction | 1.44 | Gamma | 0.63 |
| P4 | interaction | 0.39 | Gamma | 0.75 |

Oscillatory power is not modulated as a function of MH and motor impairment (UPDRS-part 3) (interaction between the two terms).

TABLE 4

| | | | FREQ | PERMUTATION |
|---|---|---|---|---|
| CHAN | EFFECT | F-VALUES | BAND | P-VALUES (FDR-COR.) |
| FP1 | interaction | 0.08 | Theta | 0.95 |
| FP2 | interaction | 0.1 | Theta | 0.95 |
| F7 | interaction | 4.95 | Theta | 0.21 |
| F3 | interaction | 1.47 | Theta | 0.66 |
| FZ | interaction | 6.79 | Theta | 0.14 |
| F4 | interaction | 0.52 | Theta | 0.82 |
| F8 | interaction | 0 | Theta | 0.98 |
| T7 | interaction | 0 | Theta | 0.98 |
| C3 | interaction | 1.48 | Theta | 0.66 |
| CZ | interaction | 0.44 | Theta | 0.82 |
| C4 | interaction | 1.07 | Theta | 0.67 |
| T8 | interaction | 6.64 | Theta | 0.14 |
| P7 | interaction | 3.01 | Theta | 0.45 |
| P3 | interaction | 0.9 | Theta | 0.67 |
| PZ | interaction | 0.34 | Theta | 0.82 |
| P4 | interaction | 0.07 | Theta | 0.95 |
| P8 | interaction | 0.02 | Theta | 0.98 |
| O1 | interaction | 1.07 | Theta | 0.67 |
| O2 | interaction | 2.22 | Theta | 0.55 |
| FP1 | interaction | 1.06 | Alpha | 0.47 |
| FP2 | interaction | 1.55 | Alpha | 0.47 |
| F7 | interaction | 0.64 | Alpha | 0.51 |
| F3 | interaction | 1.47 | Alpha | 0.47 |
| FZ | interaction | 1.06 | Alpha | 0.47 |
| F4 | interaction | 0.62 | Alpha | 0.51 |
| F8 | interaction | 0.96 | Alpha | 0.47 |
| T7 | interaction | 1.6 | Alpha | 0.47 |
| C3 | interaction | 1.52 | Alpha | 0.47 |
| CZ | interaction | 3.63 | Alpha | 0.47 |
| C4 | interaction | 1.45 | Alpha | 0.47 |
| T8 | interaction | 2.35 | Alpha | 0.47 |
| P7 | interaction | 2.48 | Alpha | 0.47 |
| P3 | interaction | 0.23 | Alpha | 0.65 |
| PZ | interaction | 0.89 | Alpha | 0.47 |
| P4 | interaction | 1.07 | Alpha | 0.47 |
| P8 | interaction | 0.37 | Alpha | 0.6 |
| O1 | interaction | 1.07 | Alpha | 0.47 |
| O2 | interaction | 0.29 | Alpha | 0.63 |
| FP1 | interaction | 3.03 | Beta | 0.16 |
| FP2 | interaction | 1.21 | Beta | 0.29 |
| F7 | interaction | 2.75 | Beta | 0.16 |
| F3 | interaction | 3.73 | Beta | 0.16 |
| FZ | interaction | 2.91 | Beta | 0.16 |
| F4 | interaction | 1.43 | Beta | 0.28 |
| F8 | interaction | 4.23 | Beta | 0.16 |
| T7 | interaction | 1.24 | Beta | 0.29 |
| C3 | interaction | 2.44 | Beta | 0.17 |
| CZ | interaction | 5.06 | Beta | 0.16 |
| C4 | interaction | 3.61 | Beta | 0.16 |
| T8 | interaction | 2.75 | Beta | 0.16 |
| P7 | interaction | 3.67 | Beta | 0.16 |
| P3 | interaction | 3.36 | Beta | 0.16 |
| PZ | interaction | 3.64 | Beta | 0.16 |
| P4 | interaction | 0.5 | Beta | 0.47 |
| P8 | interaction | 1.54 | Beta | 0.28 |
| O1 | interaction | 2.77 | Beta | 0.16 |
| O2 | interaction | 3.71 | Beta | 0.16 |
| FP1 | interaction | 0.52 | Gamma | 0.87 |
| FP2 | interaction | 0.08 | Gamma | 0.96 |
| F7 | interaction | 0.86 | Gamma | 0.71 |
| F3 | interaction | 1.18 | Gamma | 0.64 |
| FZ | interaction | 0.39 | Gamma | 0.87 |
| F4 | interaction | 0.03 | Gamma | 0.96 |
| F8 | interaction | 1.25 | Gamma | 0.64 |
| T7 | interaction | 3.71 | Gamma | 0.56 |
| C3 | interaction | 0.02 | Gamma | 0.96 |
| CZ | interaction | 3.64 | Gamma | 0.56 |
| C4 | interaction | 0.37 | Gamma | 0.87 |
| T8 | interaction | 1.85 | Gamma | 0.64 |
| P7 | interaction | 3.05 | Gamma | 0.56 |
| P3 | interaction | 0.01 | Gamma | 0.96 |
| PZ | interaction | 0 | Gamma | 0.96 |
| P4 | interaction | 0.28 | Gamma | 0.88 |
| P8 | interaction | 0.22 | Gamma | 0.88 |
| O1 | interaction | 1.73 | Gamma | 0.64 |
| O2 | interaction | 1.3 | Gamma | 0.64 |

Center frequency is modulated as a function of MH and frontal-subcortical cognitive functions (but not with posterior cognitive functions).

TABLE 5

| | | | | PERMUTATION |
|---|---|---|---|---|
| | | F- | COGNITIVE | P-VALUES |
| CHAN | EFFECT | VALUES | FUNCTIONS | (FDR-COR.) |
| FP1 | interaction | 2.78 | Frontal-subcortical | 0.12 |
| FP2 | interaction | 5.28 | Frontal-subcortical | 0.06 |
| F7 | interaction | 3.52 | Frontal-subcortical | 0.11 |
| F3 | interaction | 4.13 | Frontal-subcortical | 0.09 |
| FZ | interaction | 5.22 | Frontal-subcortical | 0.06 |
| F4 | interaction | 2.42 | Frontal-subcortical | 0.14 |
| F8 | interaction | 2.25 | Frontal-subcortical | 0.15 |
| T7 | interaction | 4.44 | Frontal-subcortical | 0.09 |
| C3 | interaction | 9.54 | Frontal-subcortical | 0.03* |
| CZ | interaction | 5.33 | Frontal-subcortical | 0.06 |
| C4 | interaction | 3.42 | Frontal-subcortical | 0.11 |
| T8 | interaction | 10.34 | Frontal-subcortical | 0.02* |
| P7 | interaction | 5.65 | Frontal-subcortical | 0.06 |
| P3 | interaction | 3.21 | Frontal-subcortical | 0.11 |
| PZ | interaction | 1.53 | Frontal-subcortical | 0.21 |
| P4 | interaction | 1.88 | Frontal-subcortical | 0.18 |
| P8 | interaction | 2.52 | Frontal-subcortical | 0.14 |
| O1 | interaction | 6.54 | Frontal-subcortical | 0.06 |
| O2 | interaction | 6.98 | Frontal-subcortical | 0.06 |
| FP1 | interaction | 2.55 | Posterior | 0.24 |
| FP2 | interaction | 2.95 | Posterior | 0.24 |
| F7 | interaction | 1.82 | Posterior | 0.25 |
| F3 | interaction | 2.39 | Posterior | 0.24 |
| FZ | interaction | 3.32 | Posterior | 0.24 |
| F4 | interaction | 1.53 | Posterior | 0.25 |
| F8 | interaction | 1.05 | Posterior | 0.34 |
| T7 | interaction | 2.45 | Posterior | 0.24 |
| C3 | interaction | 3.86 | Posterior | 0.24 |
| CZ | interaction | 5.25 | Posterior | 0.24 |
| C4 | interaction | 2.53 | Posterior | 0.24 |
| T8 | interaction | 3.05 | Posterior | 0.24 |
| P7 | interaction | 3.13 | Posterior | 0.24 |
| P3 | interaction | 1.73 | Posterior | 0.25 |
| PZ | interaction | 0.11 | Posterior | 0.75 |
| P4 | interaction | 1.6 | Posterior | 0.25 |
| P8 | interaction | 1.5 | Posterior | 0.25 |
| O1 | interaction | 2.39 | Posterior | 0.24 |
| O2 | interaction | 1.65 | Posterior | 0.25 |

MH in PD are not associated with modulations of the oscillatory power.

TABLE 6

| | | PERMUTATION | Power | Power |
|---|---|---|---|---|
| | | P-VALUES | PD-MH | PD-nMH |
| CHAN | FREQUENCY | (FDR-COR) | MEAN ± SD | MEAN ± SD |
| C3 | Theta | 0.55 | 9.1 ± 1.43 | 9.56 ± 1.48 |
| C4 | Theta | 0.2 | 8.65 ± 1.31 | 9.51 ± 1.15 |
| CZ | Theta | 0.1 | 8.85 ± 1.53 | 9.41 ± 1.08 |
| F3 | Theta | 0.26 | 8.72 ± 1.52 | 9.1 ± 1.12 |
| F4 | Theta | 0.5 | 8.8 ± 1.62 | 9.07 ± 1.11 |
| F7 | Theta | 0.1 | 8.51 ± 1.34 | 9.16 ± 1 |
| F8 | Theta | 0.1 | 8.88 ± 1.26 | 9.46 ± 1.21 |
| FP1 | Theta | 0.1 | 8.53 ± 1.27 | 9.35 ± 1.09 |
| FP2 | Theta | 0.1 | 8.66 ± 1.39 | 9.39 ± 1.11 |
| FZ | Theta | 0.22 | 8.68 ± 1.52 | 9.2 ± 1.11 |
| O1 | Theta | 0.5 | 8.37 ± 1.37 | 8.99 ± 1.09 |
| O2 | Theta | 0.44 | 8.33 ± 1.32 | 8.87 ± 0.96 |
| P3 | Theta | 0.44 | 8.35 ± 1.12 | 8.91 ± 0.96 |
| P4 | Theta | 0.72 | 8.43 ± 1.18 | 9.09 ± 1.1 |
| P7 | Theta | 0.1 | 8.54 ± 1.5 | 9.31 ± 1.13 |
| P8 | Theta | 0.56 | 8.69 ± 1.52 | 9.01 ± 1.11 |
| PZ | Theta | 0.5 | 8.68 ± 1.43 | 9.14 ± 1.04 |
| T7 | Theta | 0.5 | 8.87 ± 1.61 | 9.49 ± 1.2 |
| T8 | Theta | 0.5 | 8.49 ± 1.4 | 9.12 ± 0.9 |
| C3 | Alpha | 0.99 | 0.96 ± 0.37 | 0.96 ± 0.26 |
| C4 | Alpha | 0.99 | 1.15 ± 0.49 | 1.2 ± 0.4 |
| CZ | Alpha | 0.99 | 1.15 ± 0.5 | 1.18 ± 0.41 |
| F3 | Alpha | 0.99 | 0.95 ± 0.44 | 0.94 ± 0.39 |
| F4 | Alpha | 0.99 | 0.93 ± 0.39 | 0.93 ± 0.31 |
| F7 | Alpha | 0.99 | 0.9 ± 0.47 | 0.92 ± 0.41 |
| F8 | Alpha | 0.99 | 1.14 ± 0.55 | 1.17 ± 0.4 |
| FP1 | Alpha | 0.99 | 0.86 ± 0.44 | 0.94 ± 0.41 |
| FP2 | Alpha | 0.99 | 0.87 ± 0.43 | 0.93 ± 0.44 |
| FZ | Alpha | 0.99 | 0.99 ± 0.43 | 0.99 ± 0.39 |
| O1 | Alpha | 0.99 | 0.99 ± 0.47 | 1.02 ± 0.3 |
| O2 | Alpha | 0.99 | 1.01 ± 0.49 | 1.11 ± 0.38 |
| P3 | Alpha | 0.99 | 0.76 ± 0.43 | 0.8 ± 0.38 |
| P4 | Alpha | 0.99 | 0.79 ± 0.34 | 0.8 ± 0.31 |
| P7 | Alpha | 0.99 | 0.95 ± 0.59 | 1.08 ± 0.45 |
| P8 | Alpha | 0.99 | 0.76 ± 0.37 | 0.82 ± 0.26 |
| PZ | Alpha | 0.99 | 0.8 ± 0.44 | 0.76 ± 0.38 |
| T7 | Alpha | 0.99 | 0.93 ± 0.42 | 0.96 ± 0.3 |
| T8 | Alpha | 0.99 | 1 ± 0.57 | 1.12 ± 0.45 |
| C3 | Beta | 0.49 | 0.77 ± 0.29 | 0.83 ± 0.22 |
| C4 | Beta | 0.54 | 0.75 ± 0.27 | 0.81 ± 0.22 |
| CZ | Beta | 0.49 | 0.8 ± 0.33 | 0.88 ± 0.25 |
| F3 | Beta | 0.78 | 0.7 ± 0.31 | 0.73 ± 0.2 |
| F4 | Beta | 0.49 | 0.82 ± 0.25 | 0.91 ± 0.2 |
| F7 | Beta | 0.49 | 0.65 ± 0.27 | 0.72 ± 0.24 |
| F8 | Beta | 0.49 | 0.77 ± 0.3 | 0.84 ± 0.2 |
| FP1 | Beta | 0.49 | 0.56 ± 0.24 | 0.63 ± 0.2 |
| FP2 | Beta | 0.73 | 0.62 ± 0.25 | 0.65 ± 0.21 |
| FZ | Beta | 0.91 | 0.76 ± 0.3 | 0.77 ± 0.18 |
| O1 | Beta | 0.58 | 0.62 ± 0.3 | 0.67 ± 0.21 |
| O2 | Beta | 0.49 | 0.6 ± 0.26 | 0.67 ± 0.21 |
| P3 | Beta | 0.91 | 0.52 ± 0.22 | 0.52 ± 0.2 |
| P4 | Beta | 0.49 | 0.49 ± 0.2 | 0.55 ± 0.19 |
| P7 | Beta | 0.91 | 0.55 ± 0.26 | 0.56 ± 0.22 |
| P8 | Beta | 0.49 | 0.52 ± 0.17 | 0.58 ± 0.2 |
| PZ | Beta | 0.91 | 0.55 ± 0.24 | 0.55 ± 0.22 |
| T7 | Beta | 0.49 | 0.74 ± 0.28 | 0.83 ± 0.24 |
| T8 | Beta | 0.49 | 0.59 ± 0.28 | 0.65 ± 0.22 |
| C3 | Gamma | 0.93 | 0.2 ± 0.08 | 0.21 ± 0.09 |
| C4 | Gamma | 0.93 | 0.17 ± 0.08 | 0.21 ± 0.12 |
| CZ | Gamma | 0.93 | 0.21 ± 0.19 | 0.23 ± 0.12 |
| F3 | Gamma | 0.93 | 0.25 ± 0.24 | 0.19 ± 0.14 |
| F4 | Gamma | 0.93 | 0.25 ± 0.18 | 0.25 ± 0.14 |
| F7 | Gamma | 0.93 | 0.2 ± 0.14 | 0.2 ± 0.16 |
| F8 | Gamma | 0.93 | 0.19 ± 0.11 | 0.22 ± 0.12 |
| FP1 | Gamma | 0.93 | 0.2 ± 0.13 | 0.19 ± 0.11 |
| FP2 | Gamma | 0.93 | 0.18 ± 0.09 | 0.2 ± 0.1 |
| FZ | Gamma | 0.93 | 0.28 ± 0.21 | 0.24 ± 0.21 |
| O1 | Gamma | 0.93 | 0.15 ± 0.11 | 0.17 ± 0.1 |
| O2 | Gamma | 0.93 | 0.17 ± 0.1 | 0.17 ± 0.08 |
| P3 | Gamma | 0.93 | 0.17 ± 0.09 | 0.16 ± 0.07 |
| P4 | Gamma | 0.93 | 0.14 ± 0.06 | 0.17 ± 0.07 |
| P7 | Gamma | 0.93 | 0.16 ± 0.09 | 0.15 ± 0.11 |
| P8 | Gamma | 0.93 | 0.18 ± 0.07 | 0.18 ± 0.09 |
| PZ | Gamma | 0.93 | 0.16 ± 0.1 | 0.16 ± 0.07 |
| T7 | Gamma | 0.93 | 0.2 ± 0.14 | 0.17 ± 0.09 |
| T8 | Gamma | 0.93 | 0.18 ± 0.12 | 0.16 ± 0.12 |

TABLE 7

| | | PERMUTATION | Power | Power |
|---|---|---|---|---|
| | | P-VALUES | PD-MH | PD-nMH |
| CHAN | FREQUENCY | (FDR-COR) | MEAN ± SD | MEAN ± SD |
| C3 | Theta-Alpha | 0.2 | 9.1 ± 1.43 | 9.56 ± 1.48 |
| C4 | Theta-Alpha | 0.049* | 8.65 ± 1.31 | 9.51 ± 1.15 |
| CZ | Theta-Alpha | 0.09 | 8.85 ± 1.53 | 9.41 ± 1.08 |
| F3 | Theta-Alpha | 0.24 | 8.72 ± 1.52 | 9.1 ± 1.12 |
| F4 | Theta-Alpha | 0.39 | 8.8 ± 1.62 | 9.07 ± 1.11 |
| F7 | Theta-Alpha | 0.05 | 8.51 ± 1.34 | 9.16 ± 1 |
| F8 | Theta-Alpha | 0.08 | 8.88 ± 1.26 | 9.46 ± 1.21 |
| FP1 | Theta-Alpha | 0.05 | 8.53 ± 1.27 | 9.35 ± 1.09 |
| FP2 | Theta-Alpha | 0.05 | 8.66 ± 1.39 | 9.39 ± 1.11 |
| FZ | Theta-Alpha | 0.12 | 8.68 ± 1.52 | 9.2 ± 1.11 |
| O1 | Theta-Alpha | 0.06 | 8.37 ± 1.37 | 8.99 ± 1.09 |
| O2 | Theta-Alpha | 0.08 | 8.33 ± 1.32 | 8.87 ± 0.96 |
| P3 | Theta-Alpha | 0.06 | 8.35 ± 1.12 | 8.91 ± 0.96 |
| P4 | Theta-Alpha | 0.05 | 8.43 ± 1.18 | 9.09 ± 1.1 |
| P7 | Theta-Alpha | 0.05 | 8.54 ± 1.5 | 9.31 ± 1.13 |
| P8 | Theta-Alpha | 0.3 | 8.69 ± 1.52 | 9.01 ± 1.11 |
| PZ | Theta-Alpha | 0.12 | 8.68 ± 1.43 | 9.14 ± 1.04 |
| T7 | Theta-Alpha | 0.09 | 8.87 ± 1.61 | 9.49 ± 1.2 |
| T8 | Theta-Alpha | 0.049* | 8.49 ± 1.4 | 9.12 ± 0.9 |

Aperiodic signals are not significantly modulated by MH and frontal-subcortical cognitive functions (interaction between the two terms).

TABLE 8

| | | PERMUTATION | | |
|---|---|---|---|---|
| | APERIODIC | P-VALUES | PD-MH | PD-nMH |
| AN | SIGNAL | (FDR-COR) | MEAN ± SD | MEAN ± SD |
| C3 | Exponent | 0.89 | 1.55 ± 0.49 | 1.54 ± 0.38 |
| C4 | Exponent | 0.89 | 1.76 ± 0.29 | 1.69 ± 0.35 |
| CZ | Exponent | 0.89 | 1.75 ± 0.35 | 1.7 ± 0.38 |
| F3 | Exponent | 0.89 | 1.66 ± 0.4 | 1.62 ± 0.39 |
| F4 | Exponent | 0.89 | 1.76 ± 0.35 | 1.81 ± 0.27 |
| F7 | Exponent | 0.89 | 1.56 ± 0.42 | 1.6 ± 0.38 |
| F8 | Exponent | 0.89 | 1.88 ± 0.39 | 1.85 ± 0.33 |
| FP1 | Exponent | 0.89 | 1.56 ± 0.36 | 1.57 ± 0.36 |
| FP2 | Exponent | 0.89 | 1.58 ± 0.43 | 1.55 ± 0.4 |
| FZ | Exponent | 0.89 | 1.81 ± 0.36 | 1.77 ± 0.33 |
| O1 | Exponent | 0.89 | 1.57 ± 0.41 | 1.49 ± 0.4 |
| O2 | Exponent | 0.89 | 1.58 ± 0.35 | 1.5 ± 0.38 |
| P3 | Exponent | 0.89 | 1.52 ± 0.4 | 1.51 ± 0.37 |
| P4 | Exponent | 0.89 | 1.26 ± 0.55 | 1.23 ± 0.61 |
| P7 | Exponent | 0.89 | 1.51 ± 0.39 | 1.4 ± 0.41 |
| P8 | Exponent | 0.89 | 1.2 ± 0.63 | 1.36 ± 0.52 |
| PZ | Exponent | 0.89 | 1.56 ± 0.44 | 1.5 ± 0.42 |
| T7 | Exponent | 0.89 | 1.43 ± 0.55 | 1.5 ± 0.45 |
| T8 | Exponent | 0.89 | 1.59 ± 0.41 | 1.55 ± 0.47 |
| C3 | Offset | 0.83 | 0.51 ± 0.48 | 0.44 ± 0.44 |
| C4 | Offset | 0.83 | 0.75 ± 0.41 | 0.65 ± 0.47 |
| CZ | Offset | 0.83 | 0.73 ± 0.49 | 0.59 ± 0.47 |
| F3 | Offset | 0.83 | 0.67 ± 0.44 | 0.59 ± 0.44 |
| F4 | Offset | 0.86 | 0.71 ± 0.43 | 0.75 ± 0.4 |
| F7 | Offset | 0.97 | 0.59 ± 0.47 | 0.6 ± 0.41 |
| F8 | Offset | 0.83 | 0.8 ± 0.57 | 0.74 ± 0.43 |
| FP1 | Offset | 0.83 | 0.63 ± 0.44 | 0.54 ± 0.42 |
| FP2 | Offset | 0.83 | 0.65 ± 0.51 | 0.54 ± 0.44 |
| FZ | Offset | 0.83 | 0.77 ± 0.48 | 0.71 ± 0.44 |
| O1 | Offset | 0.83 | 0.77 ± 0.46 | 0.61 ± 0.46 |
| O2 | Offset | 0.83 | 0.75 ± 0.43 | 0.62 ± 0.47 |
| P3 | Offset | 0.83 | 0.71 ± 0.42 | 0.65 ± 0.5 |
| P4 | Offset | 0.83 | 0.51 ± 0.54 | 0.44 ± 0.6 |
| P7 | Offset | 0.83 | 0.76 ± 0.46 | 0.58 ± 0.46 |
| P8 | Offset | 0.97 | 0.44 ± 0.53 | 0.46 ± 0.48 |
| PZ | Offset | 0.83 | 0.73 ± 0.45 | 0.67 ± 0.41 |
| T7 | Offset | 0.97 | 0.37 ± 0.56 | 0.37 ± 0.46 |
| T8 | Offset | 0.83 | 0.82 ± 0.54 | 0.69 ± 0.58 |

1-38. (canceled)

39. A system for predicting a prognosis of a neuropsychological and/or a neuropsychiatric status in a subject, the system comprising:
a first device for measuring or obtaining data regarding the neuropsychiatric and/or neuropsychological status of a subject at a time T₀, said data comprising information related to Minor Hallucination (MH) events, wherein said MH events comprise sense of presence and/or passage hallucinations and/or visual illusions;
a second measuring device for measuring electrophysiological data of the subjects at a time T₀, said electrophysiological data comprising frontal theta oscillatory frequency band power and/or activity; and
a data analyzing device for gathering and analyzing the data regarding the neuropsychiatric and/or neuropsychological status and the electrophysiological data and comparing the data to a reference;
wherein a report or a presence of said Minor Hallucination (MH) events, in the absence of report or presence of Visual Hallucination (VH) events, together with an enhanced frontal theta oscillatory frequency band power and/or activity only in frontal brain regions and in the absence of alterations in alpha and/or beta and/or gamma oscillatory pattern(s), is indicative of a negative prognosis of the neuropsychological and/or neuropsychiatric status in the subject.

40. The system of claim 39, wherein the system further comprises a memory device for storing the data regarding the neuropsychiatric and/or neuropsychological status of the subject and the electrophysiological data.

41. The system of claim 39, wherein the first device comprises or consists of a robotic master-slave system configured for inducing a Presence Hallucination (PH) such as a Feeling of a Presence (FoP) hallucination.

42. The system of claim 41, wherein the robotic master-slave system comprises:
(a) a master device adapted to be moved, moved on or manipulated by the subject; and
(b) a slave device operably connected with the master device and adapted so that the subject is directly or indirectly touched by the slave device according with the master device's movement.

43. The system of claim 41, further comprising:
(c) a computer device operably connected to both the master and the slave device, the computer device being configured to modulate the time and/or space and/or force activation of the slave device in response to the activation of the master device.

44. The system of claim 39, wherein a negative prognosis of the neuropsychological and/or neuropsychiatric status comprises a higher risk of developing, and/or worsening of, a dysfunction and/or an alteration of neuropsychological and/or neuropsychiatric parameters of the subject, comprising at least one of cognitive decline, impairments or deficits in brain frontal-subcortical functions, impairments or deficits in brain posterior-cortical functions and Parkinson's Disease dementia.

45. The system of claim 39, wherein the first device measures or obtains data regarding the neuropsychological and/or neuropsychiatric status of the subject at the time T₀, and the second measuring device comprises an electroencephalogram (EEG) device.

46. The system of claim 39, wherein the reference of frontal theta oscillatory frequency band power and/or activity refers to EEG recordings data obtained from at least one healthy subject or a population thereof, a subject with no report or presence of Minor Hallucination (MH) events or a population thereof, a subject with no dysfunction and/or alteration of neuropsychological parameters or a population thereof, and a subject diagnosed with a neurological or neuropsychiatric disease or a population thereof.

47. A method for predicting a prognosis of the neuropsychological and/or neuropsychiatric status in a subject, the method comprising the steps of:
i) obtaining data regarding the neuropsychiatric status of the subject at a time T₀, said data comprising information related to Minor Hallucination (MH) events, wherein said MH events comprise sense of presence and/or passage hallucinations and/or visual illusions; and
ii) obtaining electrophysiological data of the subject at the time T₀ comprising frontal theta oscillatory frequency band power and/or activity,
wherein a report or a presence of said Minor Hallucination (MH) events in the absence of report or presence of Visual Hallucination (VH) events, together with an enhanced frontal theta oscillatory frequency band power and/or activity only in frontal brain regions and in the absence of alterations in alpha and/or beta and/or gamma oscillatory pattern(s), compared to a reference, is indicative of a negative prognosis of the neuropsychological and/or neuropsychiatric status in the subject.

48. The method of claim 47, wherein the subject is a patient suffering from a neurological disease and/or neuropsychiatric disease selected from a list comprising Alzheimer's disease, Parkinson's disease (PD), Dementia with Lewy bodies (DLB), REM sleep behaviour disorder (RBD), major depression, schizophrenia.

49. The method of claim 47, wherein a negative prognosis of the neuropsychological and/or neuropsychiatric status comprises a higher risk of developing, and/or worsening of, a dysfunction and/or an alteration of neuropsychological and/or neuropsychiatric parameters of the subject comprising at least one of cognitive decline, impairments or deficits in brain frontal-subcortical functions, impairments or deficits in brain posterior-cortical functions and Parkinson's Disease dementia.

50. The method of any one of claim 47, further comprising a step of obtaining data regarding the neuropsychological and/or neuropsychiatric status of the subject at the time T₀.

51. The method of claim 47, wherein electrophysiological data of the subject are obtained by electroencephalography (EEG) and/or through electroencephalography (EEG) recordings.

52. The method of claim 47, wherein the reference of frontal theta oscillatory frequency band power and/or activity refers to EEG recordings data obtained from at least one healthy subject or a population thereof, a subject with no report or presence of Minor Hallucination (MH) events or a population thereof, a subject with no dysfunction and/or alteration of neuropsychological parameters or a population thereof, and a subject diagnosed with a neurological or neuropsychiatric disease or a population thereof.

53. The method of claim 47, further comprising a step of obtaining data regarding the neuropsychological and/or neuropsychiatric status of the subject at a time Tᵢ, wherein Tᵢ is comprised between 1 month and 20 years from To.

54. The method of claim 47, wherein the method is a computer-implemented method.

55. A data processing apparatus comprising a processor configured to perform the method of claim 47.

56. A computer-readable data carrier having stored thereon the computer program comprising instructions which, when the program is executed by a processing apparatus/device/system, cause the system of claim 1 to carry out the steps of the method of claim 47.

57. A database comprising:
i) data regarding the neuropsychiatric and/or neuropsychological status of subjects at a time T₀, said data comprising information related to Minor Hallucination (MH) events; and
ii) electrophysiological data of the subjects at a time T₀ comprising frontal theta oscillatory frequency band power and/or activity.
